(19) United States
(12) Patent Application Publication
(10) Pub. No.: US 2024/0180220 A1
(43) Pub. Date: Jun. 6, 2024

(54) HYDROGEL ENCAPSULATIONS AND METHODS OF MAKING THE SAME

(71) Applicant: INTERNATIONAL FLAVORS & FRAGRANCES INC., NEW YORK, NY (US)

(72) Inventors: AMIR MALAKI, UNION BEACH, NJ (US); ADAM TOTH, UNION BEACH, NJ (US); RONALD GABBARD, UNION BEACH, NJ (US)

(21) Appl. No.: 18/552,935

(22) PCT Filed: Apr. 15, 2022
(86) PCT No.: PCT/US2022/025091

§ 371 Date: Sep. 28, 2023

Related U.S. Application Data
(60) Provisional application No. 63/175,850, filed on Apr. 16, 2021.

(51) Int. Cl. A23L 27/00; A23J 3/14; A23P 10/30

(52) U.S. Cl. CPC A23L 27/72; A23J 3/14; A23L 27/88; A23P 10/30

(57) ABSTRACT

The present invention relates to hydrogel encapsulation compositions capable of retaining active compounds in finished products, the finished products containing the hydrogel encapsulations, and methods relating to the same. The present invention also relates to hydrogel particles and methods relating to the same.

### FIELD OF THE INVENTION

Some embodiments of these inventions relate to encapsulation systems involving hydrogels and the methods of making those hydrogels. Some embodiments relate to the use of proteins for hydrogel gelation, and some embodiments include active ingredients in the inventive hydrogels. In still other embodiments, the active ingredients are flavor compounds that can survive comestible processing as a result of being encapsulated in the hydrogels to provide desirable organoleptic qualities to end products.

### BACKGROUND OF THE INVENTION

Various encapsulation systems such as those involving chemical cross linking or those involving absorption, adsorption, etc. are known. These encapsulation systems have varying degrees of efficacy and efficiency. Some involve relatively low levels of encapsulant while others offer incomplete encapsulation which can expose actives to forces that cause degradation.

For example, WO 2014/064591 describes a microencapsulation of omega-3 fatty acids using legume proteins where a relatively low level of actives are emulsified with protein by mixing a solution of the actives and proteins overnight at a low temperature to create a microcapsule that can be released in the digestive system of a mammal. Such low temperatures do not enable protein gelation and thus the microcapsule will have limited volatile retention. Also, because of the relatively low level of actives, larger quantities of the microcapsules are needed which can be difficult for finished product formulations.

For actives where a larger amount of the active needs to be included in the finished product system and protected from the rigors of finished product processing, however, these known methods fall short.

Thus, there is a need for an encapsulation system that can survive finished product processing and deliver actives to a consumer with better efficacy and efficiency.

### SUMMARY OF THE INVENTION

In some embodiments, a method of encapsulation comprises the steps of homogenizing an emulsifier with an active and a carrier to create an emulsified active, heating the emulsified active to a first temperature of at least 35 C, mixing the heated emulsified active with a protein, and further heating the mixture to a second temperature of at least 50 C to create a hydrogel. In some embodiments, the method further comprises the additional step of holding the hydrogel at the second temperature for a gelation time of at least 30 minutes. In other embodiments, the encapsulation method further comprises the step of drying the hydrogel to create hydrogel particles.

In some embodiments, the protein comprises at least one of a chickpea protein, a lentil protein, a faba bean protein, a soybean protein, or combinations thereof. In some preferred embodiments, the protein is a faba bean protein and in some particularly preferred embodiments, the faba bean protein is unhydrolyzed.

In some embodiments, the amount of protein in the hydrogel is at least 7% w/v by weight of the hydrogel and in other embodiments, the protein is present in an amount of from about 7% to about 20% w/v by weight of the hydrogel.

In some embodiments involving the drying step, the protein is present in the hydrogel particles at an amount of from about 10% to about 50% w/w by weight of the hydrogel particles. In some embodiments, the active in the hydrogel is present in an amount of from about 0.1% to about 60% w/v by weight of the hydrogel. And in embodiments with a drying step, the active is present in amounts of from about 0.1% to about 60% w/w by weight of the hydrogel particles.

In some embodiments, a microcapsule comprises an active and a shell, wherein the active comprises a flavor, the shell comprises a faba bean protein, and the microcapsule has a volatile retention of at least 80%. In some embodiments the microcapsule has a volatile retention of at least 90%.

In some embodiments, a product comprises a product base and a microcapsule, wherein the microcapsule comprises an active and a shell, wherein the active comprises a flavor, the shell comprises a faba bean protein, and the microcapsule has a volatile retention of at least 80%. In other embodiments, the microcapsule has a volatile retention of 90%. In some of these embodiments, the active is present in the microcapsule in an amount of from about 0.1% to about 60% w/w by weight of the microcapsule. In others of these embodiments, the microcapsule is present in the product in an amount of from about 0.1% to about 10% w/w by weight of the product.

### Overview

1. A method of encapsulation comprising the steps of homogenizing an emulsifier with an active and a carrier to create an emulsified active, heating the emulsified active to a first temperature of at least 35 C, mixing the heated emulsified active with a protein, and further heating the mixture to a second temperature of at least 50 C to create a hydrogel.
2. The method as in claim 1, further comprising a step of holding the hydrogel at the second temperature for a gelation time of at least 30 minutes.
3. The method as in claim 2, wherein the hydrogel has a viscosity of at least 300 Cps at 30 C.
4. The method as in claim 1, wherein the hydrogel has a particle size of less than 30 microns.
5. The method as in claim 1, wherein the hydrogel has a particle size of from about 0.5 microns to about 20 microns.
6. The method as in claim 1, wherein the emulsified active has a particle size of less than 10 microns.
7. The method as in claim 1, further comprising a step of cooling the hydrogel to a cooling temperature of from about 20 C to about 70 C.
8. The method as in claim 1, further comprising a step of drying the hydrogel to create hydrogel particles.
9. The method as in claim 8, wherein the hydrogel particles have a particle size of from about 2 microns to about 500 microns.
10. The method as in claim 8, wherein the drying step comprises at least one of spray drying, spray chilling, fluidized bed drying, drum drying, freeze drying, vacuum drying, vacuum freeze drying, or combinations thereof.
11. The method as in claim 1, wherein the emulsifier comprises at least one of a lecithin, a sucrose ester, a protein, a modified food starch, a gum, a gum acacia, a soap-bark extract, a saponin, or combinations thereof.
12. The method as in claim 1, wherein the emulsifier comprises a blend of at least two emulsifiers.
13. The method as in claim 1, wherein the active comprises at least one of a fragrance, a pro-fragrance, a flavor, a malodor counteractive agent, a vitamin, a vitamin derivative, an anti-inflammatory agent, a fungicide, an anesthetic, an analgesic, an antimicrobial active, an anti-viral agent, an anti-infectious agent, a pharmaceutical agent, a nutraceutical agent, an anti-acne agent, a skin lightening agent, an insect repellent, an animal repellent, a vermin repellent, an emollient, a skin moisturizing agent, a wrinkle control agent, a UV protection agent, a fabric softener active, a hard surface cleaning active, a skin conditioning agent, a hair conditioning agent, a flame retardant, an antistatic agent, a taste modulator, a cell, a probiotic, a colorant, a vegetable oil, a fish oil, or combinations thereof.
14. The method as in claim 1, wherein the carrier comprises at least one of an inulin, a maltodextrin, a glycose syrup solid, a maltose, a modified food starch, a dextrin, a sucrose, a fructose, a polyol, a vegetable fiber, a salt, or combinations thereof.
15. The method as in claim 1, wherein the carrier is present in an amount of from about 10% to about 30% w/v by weight of the hydrogel.
16. The method as in claim 8, wherein the carrier is present in an amount of from about 15% to about 60% w/w by weight of the hydrogel particle.
17. The method as in claim 1, wherein the protein comprises at least one of a chickpea protein, a lentil protein, a faba bean protein, a soybean protein, or combinations thereof.
18. The method as in claim 1, wherein the protein is a faba bean protein.
19. The method as in claim 18, wherein the faba bean protein is unhydrolyzed.
20. The method as in claim 1, wherein the protein has a protein content of at least 85% w/w by weight of the protein.
21. The method as in claim 1, wherein the method further comprises mixing the active with a weighting agent prior to the homogenization step.
22. The method as in claim 21, wherein the weighting agent comprises at least one of a sucrose acetate isobutyrate, an ester gum, or combinations thereof.
23. The method as in claim 1, wherein the homogenization step further comprises an antioxidant.
24. The method as in claim 23, wherein the antioxidant comprises at least one of a carotenoid, a vitamin, BHA, BHT, TBHQ, propyl gallate, a polyphenolic, a glucosinolate, rosemary extract, or combinations thereof.
25. The method as in claim 1, wherein the mixing step further comprises an acidulant.
26. The method as in claim 25, wherein the acidulant comprises at least one citric acid, malic acid, acetic acid, phosphoric acid, tartaric acid, sulfuric acid, glucono delta lactone, or combinations thereof.
27. The method as in claim 1, wherein the active is present in an amount of from about 0.1% to about 60% w/v by weight of the hydrogel.
28. The method as in claim 8, wherein the active is present in an amount of from about 0.1% to about 60% w/w by weight of the hydrogel particles.
29. The method as in claim 1, wherein the protein is present in an amount of at least 7% w/v by weight of the hydrogel.
30. The method as in claim 1, wherein the protein is present in an amount of from about 7% to about 20% w/v by weight of the hydrogel.
31. The method as in claim 30, wherein the emulsifier is present in an amount of from about 5% to about 20% w/v by weight of the hydrogel.
32. The method as in claim 8, wherein the protein is present in an amount of from about 10% to about 50% w/w by weight of the hydrogel particle.
33. The method as in claim 1, wherein the emulsifier is present in an amount of from about 5% to about 20% w/v by weight of the hydrogel.
34. The method as in claim 8, wherein the emulsifier is present in an amount of from about 10% to about 50% w/w by weight of the hydrogel particle.
35. A method of improving the heat stability of an active comprising the steps of homogenizing an emulsifier with the active and a carrier to create an emulsified active, heating the emulsified active to a first temperature of at least 35 C, mixing the heated emulsified active with a protein, and heating the mixture to a second temperature of at least 50 C to create a hydrogel; wherein the active exhibits heat stability.
36. The method as in claim 35, wherein heat stability is measured by at least one of volatile retention, time intensity, or combinations thereof.
37. The method as in claim 35, the method further comprising a step of holding the mixture of heated emulsified active and protein at the second temperature for a gelation time of at least 30 minutes.
38. The method as in claim 35, further comprising a step of cooling the hydrogel to a cooling temperature of from about 20 C to about 70 C.
39. The method as in claim 35, further comprising a step of drying the hydrogel to create hydrogel particles.
40. An encapsulation made from the method as in claim 1.
41. A microcapsule comprising an active and a shell obtainable by any one of the claims 1 to 34, wherein the active comprises a flavor and the shell comprises a faba bean protein, and wherein the microcapsule has a volatile retention of at least 80%.
42. The microcapsule of claim 41 wherein the microcapsule has a volatile retention of at least 90%.
43. A method of encapsulation comprising the steps of:
   i. Dissolving an emulsifier and a carrier in a quantity of water to form an aqueous matrix;
   ii. Homogenizing a flavor with the aqueous matrix to form a flavor emulsion;
   iii. Heating the flavor emulsion to a first temperature of at least 50 C;
   iv. Mixing a protein into the heated flavor emulsion to create a mixture;
   V. Heating the mixture to a second temperature of at least 70 C;
   vi. Holding the mixture at the second temperature for a gelation time of at least 30 minutes to create a hydrogel;
   vii. Cooling the hydrogel to a cooling temperature of from about 20 C to about 70 C; and
   viii. Drying the cooled hydrogel to create hydrogel particles.
44. The method as in claim 43, wherein the hydrogel particles have a mean particle size of from about 120 microns to about 130 microns.
45. The method as in claim 43, wherein the hydrogel particles have a volatile retention of at least 80%.
46. The method of claim 43, wherein the hydrogel particle has a volatile retention of at least 90%.
47. The method as in claim 43, wherein the hydrogel particle contains an amount of flavor from about 0.1% to about 60% w/w by weight of the hydrogel particle.
48. The method as in claim 43, wherein the hydrogel contains an amount of flavor from about 0.1% to about 60% w/v by weight of the hydrogel.
49. The method as in claim 43, wherein the emulsifier comprises at least one of a lecithin, a sucrose ester, a protein, a modified food starch, a gum, a gum acacia, a soap-bark extract, a saponin, or combinations thereof.
50. The method as in claim 43, wherein the emulsifier comprises at least two emulsifiers.
51. The method as in claim 43, wherein the carrier comprises at least one of an inulin, a maltodextrin, a glycose syrup solid, a maltose, a modified food starch, a dextrin, a sucrose, a fructose, a polyol, a vegetable fiber, a salt, or combinations thereof.
52. The method as in claim 43, wherein the flavor comprises at least one of a fruit flavor, a savory flavor, a dairy flavor, a bakery flavor, a reaction flavor, a taste modifier, a sweetness modifier, a cooling agent, a warming agent, a flavor enhancer, or combinations thereof.
53. The method as in claim 43, further comprising dissolving a weighting agent in the flavor prior to the homogenization step.
54. The method as in claim 53, wherein the weighting agent comprises at least one of a sucrose acetate isobutyrate, an ester gum, or combinations thereof.
55. The method as in claim 43, wherein the protein comprises at least one of a chickpea protein, a lentil protein, a faba bean protein, a soybean protein, or combinations thereof.
56. The method as in claim 43, wherein the protein is a faba bean protein.
57. The method as in claim 56, wherein the faba bean protein has a protein content of at least 85% w/w by weight of the faba bean protein.
58. The method as in claim 56, wherein the faba bean protein is unhydrolyzed.
59. The method as in claim 43, further comprising the step of adding an acidulant to the protein prior to mixing the protein into the heated flavor emulsion.
60. The method as in claim 59, wherein the acidulant comprises at least one of citric acid, malic acid, acetic acid, phosphoric acid, tartaric acid, sulfuric acid, glucono delta lactone, or combinations thereof.
61. The method as in claim 59, wherein the pH after adding the acidulant is from about 4.5 to about 5.5.
62. The method as in claim 43, wherein the drying step comprises at least one of spray drying, spray chilling, fluidized bed drying, drum drying, freeze drying, or combinations thereof.
63. An encapsuled flavor made from the method as in claim 43.
64. A microcapsule comprising an active and a shell wherein the active comprises a flavor, the shell comprises a faba bean protein, and the microcapsule has a volatile retention of at least 80%.
65. The microcapsule of claim 64 wherein the microcapsule has a volatile retention of at least 90%
66. The microcapsule as in claim 64, wherein the flavor is present in an amount of from about 0.1% to about 60% w/w by weight of the microcapsule.
67. A product comprising a product base and a microcapsule wherein the microcapsule comprises an active and a shell, wherein the shell comprises a faba bean protein, and wherein the microcapsule has a volatile retention of at least 80%.
68. The product as in claim 67, wherein the active comprises at least one of a fragrance, a pro-fragrance, a flavor, a malodor counteractive agent, a vitamin, a vitamin derivative, an anti-inflammatory agent, a fungicide, an anesthetic, an analgesic, an antimicrobial active, an anti-viral agent, an anti-infectious agent, a pharmaceutical agent, a nutraceutical agent, an anti-acne agent, a skin lightening agent, an insect repellant, an animal repellent, a vermin repellent, an emollient, a skin moisturizing agent, a wrinkle control agent, a UV protection agent, a fabric softener active, a hard surface cleaning active, a skin conditioning agent, a hair conditioning agent, a flame retardant, an antistatic agent, a taste modulator, a cell, a probiotic, a colorant, a vegetable oil, a fish oil, or combinations thereof.
69. The product as in claim 68, wherein the flavor comprises at least one of a fruit flavor, a savory flavor, a dairy flavor, a bakery flavor, a reaction flavor, a taste modifier, a sweetness modifier, a cooling agent, a warming agent, a flavor enhancer, or combinations thereof.
70. The product as in claim 69, wherein the product has a higher flavor intensity score as measured by sensory testing than an iso-active flavored product without the microcapsule.
71. The product as in claim 69, wherein the flavor is present in the microcapsule in an amount of from about 0.1% to about 60% w/w by weight of the microcapsule.
72. The product as in claim 67, wherein the microcapsule is present in an amount of from about 0.1% to about 10% w/w by weight of the product.
73. The product as in claim 67, wherein the product base comprises at least one of a food product base, a pharmaceutical product base, a cosmetic product base, a consumer product base, or combinations thereof.
74. The product as in claim 73, wherein the food product base comprises at least one of a chewing gum, a confection, a beverage, a snack, a dairy product, a soup, a sauce, a condiment, a cereal, a baked good, or combinations thereof.
75. The product as in claim 73, wherein the consumer product base comprises at least one of an oral care product, a detergent, a fabric softener, a fabric care product, an antiperspirant, a deodorant, a talcum powder, a kitty litter, a hair care product, a styling product, a personal care product, an air freshener, a cleaner, or combinations thereof.
76. The product as in claim 73, wherein the consumer product base comprises a pet food, pet treat or pet oral care product.
77. The product as in claim 73, wherein the microcapsule has a volatile retention of at least 90%.
78. The method as in claim 1, wherein the method further comprises the step of mixing the emulsified active with a gelling agent, a cross-linking agent or a combination thereof prior to mixing the emulsified active with the protein.
79. The method as in claim 78, wherein the method further comprises the step of heating the mixture of the emulsified active and the gelling agent, cross-linking agent or combination thereof to a temperature of 25 C to 60 C.
80. A method of encapsulation comprising the steps of homogenizing an emulsifier with an active and a carrier to create an emulsified active, mixing the emulsified active with a protein to create a mixture, and mixing the mixture with an alkaline solution having a pH greater than 7.0 to create a hydrogel.

### DETAILED DESCRIPTION OF THE INVENTION

To provide an encapsulation with improved efficacy and efficiency, these inventors have designed encapsulations, interchangeably referred to herein as microcapsules, and methods to make encapsulations that involve heating proteinaceous emulsions to gelation temperatures thus creating hydrogels. These hydrogels have desirable levels of actives which can be referred to as the loading of actives and good stability in various finished products. As used herein, “loading” refers to the relative amount of an active ingredient expressed as a percentage of the encapsulation system.

It is to be understood that these inventions are not limited to the particular embodiments described. It is also to be understood that the terminology used herein is for the purpose of describing particular embodiments only, and is not intended to be limiting, since the scope of these inventions will be limited only by the appended claims.

Where a range of values is provided, it is understood that each intervening value is included, between the upper and lower limit of that range and any other stated or intervening value in that stated range, is encompassed within the invention, unless the context clearly dictates otherwise. The upper and lower limits of any smaller ranges may independently be included in the smaller ranges and are also encompassed within the inventions, subject to any specifically excluded limit in the stated range. Where the stated range includes one or both of the upper and lower limits, ranges excluding either or both of those included limits are also included in the invention.

Certain ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present inventions, representative illustrative methods and materials are herein described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present inventions are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

As will be apparent to those of skill in the art upon reading this disclosure, each of the individual embodiments described herein has discrete components and features which may be readily separated from or combined with the features of any of the other several embodiments without departing from the scope or spirit of the present inventions. Any recited method can be carried out in the order of events recited or in any other order which is logically possible.

In some embodiments, a method of encapsulation comprises the steps of homogenizing an emulsifier with an active and a carrier to create an emulsified active. That emulsified active is then heated to a first temperature of at least 35 C, after which the heated emulsified active is mixed with a protein and the mixture is heated to a second temperature of at least 50 C to create a hydrogel. As used herein, the term “hydrogel” means a composition where the protein has become sufficiently hydrated and denatured so as to create a three-dimensional network structure to entrap and retain the active.

In some embodiments, the first temperature is at least 36 C, at least 37 C, at least 38 C, at least 39 C, at least 40 C, at least 41 C, at least 42 C, at least 43 C, at least 44 C, at least 45 C, at least 46 C, at least 47 C, at least 48 C, or at least 49 C. In still other embodiments, the first temperature is from about 35 C to about 49 C. And in still other embodiments, the first temperature is from about 35 C, 36 C, 37 C, 38 C, 39 C, 40 C, 41 C, 42 C, 43 C, 44 C, 45 C, 46 C, 47 C, or 48 C to about 36 C, 37 C, 38 C, 39 C, 40 C, 41 C, 42 C, 43 C, 44 C, 45 C, 46 C, 47 C, 48 C, or 49 C.

In some embodiments, the second temperature is at least 55 C, at least 60 C, at least 65 C, at least 70 C, at least 75 C, at least 80 C, at least 85 C, at least 90 C, at least 95 C, or at least 100 C. In other embodiments, the second temperature is from about 50 C to about 100 C. In still other embodiments, the second temperature is from about 50 C, 55 C, 60 C, 65 C, 70 C, 75 C, 80 C, 85 C, 90 C, or 95 C to about 55 C, 60 C, 65 C, 70 C, 75 C, 80 C, 85 C, 90 C, 95 C, or 100 C.

In some embodiments, the method of encapsulation further comprises the step of holding the hydrogel at the second temperature for a gelation time of at least 30 minutes. In some embodiments, the gelation time is at least 35 minutes, at least 40 minutes, at least 45 minutes, at least 50 minutes, at least 55 minutes, or at least 60 minutes. In other embodiments, the gelation time is from about 30 minutes to about 60 minutes. In still other embodiments, the gelation time is from about 30 minutes, 35 minutes, 40 minutes, 45 minutes, 50 minutes, or 55 minutes to about 35 minutes, 40 minutes, 45 minutes, 50 minutes, 55 minutes, or 60 minutes.

In some embodiments, a method of encapsulation comprises the steps of homogenizing an emulsifier with an active and a carrier to create an emulsified active. That emulsified active is mixed with a protein at room temperature and subjected to alkaline conditions to create a hydrogel. The term “alkaline conditions” means a pH of greater than 7.0.

Another useful processing metric for the hydrogel is particle size of the oil droplets in the both the emulsified active and in the hydrogel. The oil droplet particle size metric can be measured by using a Beckman Coulter LS 13 320 Laser Diffraction Particle Size Analyzer. Distilled water is used as the dispersant. Particle size distributions are calculated by the instrument according to the Fraunhofer theory. Particle size measurements are reported as volume-surface mean diameters or D. In some embodiments, the hydrogel has a particle size of less than 30 microns. In some embodiments, the hydrogel has a particle size of from about 0.5 microns to about 20 microns. In other embodiments, the hydrogel has a particle size of from about 0.5 to about 5 microns, and in other embodiments, the hydrogel has a particle size of from about 1 to about 10 microns. And in still other embodiments, the hydrogel has a particle size of from about 5 to about 20 microns. In some embodiments, the emulsified active has a particle size of less than 10 microns.

Without wishing to be bound by any theory, these inventors have shown that forming a hydrogel by heating a protein-containing matrix to a gelation temperature and holding that matrix for a gelation time creates an encapsulation that has superior performance in finished products. While pharmaceutical researchers have shown protein-containing hydrogels to be useful in releasing actives at body temperature or under gut acid conditions (see Panahi R., Baghban-Salehi M. (2019) Protein-Based Hydrogels. In: Mondal M. (eds) Cellulose-Based Superabsorbent Hydrogels. Polymers and Polymeric Composites: A Reference Series. Springer, Cham. https://doi.org/10.1007/978-3-319-77830-3_52), these inventors have surprisingly demonstrated that protein-containing hydrogels can endure much higher temperatures such as those encountered in commercial baking applications (100 C or more). As a result, finished products like bread containing protein hydrogel-encapsulated flavor compounds are perceived to have a higher intensity of the encapsulated flavor than when the flavor is not encapsulated in a protein-containing hydrogel.

In some embodiments, the method of encapsulation further comprises a step of cooling the hydrogel to a cooling temperature of from about 20 C to about 70 C. In some embodiments, the cooling temperature is from about 20 C, 21 C, 22 C, 23 C, 24 C, 25 C, 26 C, 27 C, 28 C, 29 C, 30 C, 31 C, 32 C, 33 C, 34 C, 35 C, 36 C, 37 C, 38 C, 39 C, 40 C, 41 C, 42 C, 43 C, 44 C, 45 C, 46 C, 47 C, 48 C, 49 C, 50 C, 51 C, 52 C, 53 C, 54 C, 55 C, 56 C, 57 C, 58 C, 59 C, 60 C, 61 C, 62 C, 63, 64 C, 65 C, 66 C, 67 C, 68 C, or 69 C to about 21 C, 22 C, 23 C, 24 C, 25 C, 26 C, 27 C, 28 C, 29 C, 30 C, 31 C, 32 C, 33 C, 34 C, 35 C, 36 C, 37 C, 38 C, 39 C, 40 C, 41 C, 42 C, 43 C, 44 C, 45 C, 46 C, 47 C, 48 C, 49 C, 50 C, 51 C, 52 C, 53 C, 54 C, 55 C, 56 C, 57 C, 58 C, 59 C, 60 C, 61 C, 62 C, 63, 64 C, 65 C, 66 C, 67 C, 68 C, 69 C, or 70 C.

In some embodiments, the hydrogel has a viscosity during the cooling step of at least 300 centipoise (Cps) when cooled to 30 C. This viscosity can be a useful processing indicator to signal that the hydrogel has reached a point where the encapsulation will adequately protect the active. In some embodiments, the hydrogel has a viscosity of at least 300 Cps, 400 Cps, 500 Cps, 600 Cps, 700 Cps, 800 Cps, 900 Cps, 1000 Cps, 1100 Cps, 1200 Cps, 1300 Cps, 1400 Cps, or at least 1500 Cps. In some embodiments, the hydrogel has a viscosity at 30 C of from about 300 Cps to about 2000 Cps. And in still other embodiments, the hydrogel has a viscosity of from about 300 Cps, 400 Cps, 500 Cps, 600 Cps, 700 Cps, 800 Cps, 900 Cps, 1000 Cps, 1100 Cps, 1200 Cps, 1300 Cps, 1400 Cps, 1500 Cps, 1600 Cps, 1700 Cps, 1800 Cps, or 1900 Cps to about 1100 Cps, 1200 Cps, 1300 Cps, 1400 Cps, 1500 Cps, 1600 Cps, 1700 Cps, 1800 Cps, 1900 Cps, or about 2000 Cps.

In some embodiments, the method of encapsulation further comprises a step of drying the hydrogel to create hydrogel particles. In some embodiments, the drying step comprises at least one of spray drying, spray chilling, fluidized bed drying, drum drying, infrared drying, wipe film evaporator drying, freeze drying, vacuum drying, vacuum freeze drying, or combinations thereof.

In some embodiments, the method of encapsulation further comprises the steps of milling and sieving the hydrogel particles. Those of skill in the art will be familiar with the types of equipment needed for these steps. Milling techniques can include, but are not limited to, air classifying milling, ball milling, conical milling, hammer milling, jet milling, or pin milling.

After drying and optionally milling and sieving, in some embodiments, the hydrogel particles have a particle size of from about 2 microns to about 500 microns. To measure the particle size of the hydrogel particles, a Mastersizer 3000 laser light scattering instrument (Malvern Instruments Ltd., Worcestershire, United Kingdom) equipped with a powder sample handling unit is used. Particle size distributions are calculated by the instrument according to the Fraunhofer theory. Particle size measurements are reported as volume-surface mean diameters or D. In other embodiments, the hydrogel particles have a particle size of from about 10 to about 100 microns, and in other embodiments, the hydrogel particles have a particle size of from about 50 to about 250 microns. And in still other embodiments, the hydrogel particles have a particle size of from about 100 to about 350 microns, and in other embodiments, the hydrogel particles have a particle size of from about 150 to about 450 microns, and in still other embodiments, the hydrogel particles have a particle size of from about 120 microns to 130 microns.

Those of skill in the art will be familiar with the types of equipment needed for these steps of homogenizing, heating, mixing, holding, and cooling. Homogenizers can include, but are not limited to, single stage homogenizers, two stage homogenizers, batch homogenizers, and continuous homogenizers. Similarly, conventional heating equipment such as jacketed kettles and swept surface heat exchangers can be used. Further, conventional mixing equipment like planetary mixers, paddle mixers, rotary mixers, and sigma blade mixers can be used. As to holding and cooling equipment, conventional kettles, tanks, pipes, lines, chillers, and the like can be used.

As to the emulsifier involved in the method of encapsulation, suitable emulsifiers, also known as surfactants, can include, but are not limited to lecithins, sucrose esters, proteins, food starches such as a modified food starch, gums, hydrocolloids, soap-bark extracts, saponins, and the like. These emulsifiers operate to stabilize the active during the steps of the methods. In some embodiments, the emulsifier comprises at least one of a lecithin, a sucrose ester, a protein, a modified food starch, a gum, a gum acacia, a soap-bark extract, a saponin, or combinations thereof. In some embodiments, the emulsifier comprises a blend of at least two emulsifiers.

In some embodiments, the emulsifier is present in an amount of from about 5% to about 20% w/v by weight of the hydrogel. In some embodiments, the emulsifier is present in an amount of from about 5%, 6%, 7%, 8%, 9%, 10%, 11%, 12%, 13%, 14%, 15%, 16%, 17%, 18%, or 19% to about 6%, 7%, 8%, 9%, 10%, 11%, 12%, 13%, 14%, 15%, 16%, 17%, 18%, 19%, or 20% w/v by weight of the hydrogel.

In some embodiments involving a drying step, the emulsifier is present is an amount of from about 10% to about 50% w/w by weight of the hydrogel particle. In other embodiments, the emulsifier is present in an amount of from about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49% to about 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w by weight of the hydrogel particle.

In some embodiments, the ratio of active to emulsifier is from about 2:1 to about 1:1.

As to the active involved in the method of encapsulation, a wide range of materials can be involved. Some examples include, but are not limited to, edible materials such as flavors, taste modifiers, colors, vegetable oils, and edible functional materials like vitamins, minerals, nutraceuticals, fish oils, probiotics, anti-inflammatories, or pharmaceuticals. In some embodiments, the active can be fragrance, a pro-fragrance, or an odor counteractant. In still other embodiments, the active can be a fungicide, an anesthetic, an analgesic, an antimicrobial active, an anti-viral agent, an anti-infectious agent, an anti-acne agent, a skin lightening agent, an insect repellant, an animal repellent, a vermin repellent, an emollient, a skin moisturizing agent, a wrinkle control agent, a UV protection agent, a fabric softener active, a hard surface cleaning active, a skin conditioning agent, a hair conditioning agent, a flame retardant, or an antistatic agent. In still other embodiments, the active comprises at least one of a fragrance, a pro-fragrance, a flavor, a malodor counteractive agent, a vitamin, a vitamin derivative, an anti-inflammatory agent, a fungicide, an anesthetic, an analgesic, an antimicrobial active, an anti-viral agent, an anti-infectious agent, a pharmaceutical agent, a nutraceutical agent, an anti-acne agent, a skin lightening agent, an insect repellant, an animal repellent, a vermin repellent, an emollient, a skin moisturizing agent, a wrinkle control agent, a UV protection agent, a fabric softener active, a hard surface cleaning active, a skin conditioning agent, a hair conditioning agent, a flame retardant, an antistatic agent, a taste modulator, a cell, a probiotic, a colorant, a vegetable oil, a fish oil, or combinations thereof.

In some embodiments, the active is present in the hydrogel in an amount of from about 0.1% to about 60% w/v by weight of the hydrogel. In some embodiments, the active is present in an amount of from about 0.1% to about 10% w/v by weight of the hydrogel, while in other embodiments, the active is present in an amount of from about 5% to about 25% w/v by weight of the hydrogel. In still other embodiments, the amount of active in the hydrogel is from about 20% to about 40% w/v by weight of the hydrogel and in yet other embodiments, the amount of active is from about 30% to about 50% w/v by weight of the hydrogel. In some embodiments, the amount of active in the hydrogel is from about 25% to about 60% w/v by weight of the hydrogel. In some embodiments, the amount of active in the hydrogel is from about 25%, 30%, 35%, 40%, 45%, 50%, or 55% to about 30%, 35%, 40%, 45%, 50%, 55%, or 60% w/v by weight of the hydrogel.

In embodiments involving a drying step, the active is present in the hydrogel particle in an amount of from about 0.1% to about 60% w/w by weight of the hydrogel particle. In some embodiments, the active is present in an amount of from about 0.1% to about 10% w/w by weight of the hydrogel particle, while in other embodiments, the active is present in an amount of from about 5% to about 25% w/w by weight of the hydrogel particle. In still other embodiments, the amount of active in the hydrogel particle is from about 20% to about 40% w/w by weight of the hydrogel particle and in yet other embodiments, the amount of active is from about 30% to about 50% w/w by weight of the hydrogel particle. In some embodiments, the amount of active in the hydrogel particle is from about 20% to about 60% w/w by weight of the hydrogel particle. In some embodiments, the amount of active in the hydrogel particle is from about 20%, 21%, 22%, 23%, 24%, 25%, 30%, 35%, 40%, 45%, 50%, or 55% to about 21%, 22%, 23%, 24%, 25%, 26%, 27%, 28%, 29%, 30%, 35%, 40%, 45%, 50%, 55%, or 60% w/w by weight of the hydrogel particle.

Regarding the carrier involved in the method of encapsulation, the carrier can include any sugar, sugar derivative, modified starch, protein, cellulose, salt, dextrin, gum, sugar alcohol, polyol, peptide, acid, carbohydrate or hydrocolloid that can function to enhance processing. In some embodiments, the carrier can include sugars such as sucrose, glucose, lactose, levulose, trehalose, fructose, maltose, ribose, dextrose, isomalt, sorbitol, mannitol, xylitol, lactitol, maltitol, pentatol, arabinose, pentose, xylose, galactose; hydrogenated starch hydrolysates; maltodextrins or dextrins (soluble fiber); hydrocolloids such as agar or carrageenan; gums; polydextrose; proteins such as soy and whey protein isolates and hydrolyzates, and sodium caseinates; and derivatives and mixtures thereof. In some embodiments, the carrier can be selected based upon, amongst other factors, the desired flavor, authentic taste and intensity to be achieved. In still other embodiments, carrier comprises at least one of an inulin, a maltodextrin, a glycose syrup solid, a maltose, a modified food starch, a dextrin, a sucrose, a fructose, a polyol, a vegetable fiber, a salt, or combinations thereof.

In some embodiments, the carrier is present in an amount of from about 10% to about 30% w/v by weight of the hydrogel. In other embodiments, the carrier is present in an amount of from about 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, or 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5% to about 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5% 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, or 30% w/v by weight of the hydrogel.

In some embodiments involving a drying step, the carrier is present in an amount of from about 15% to about 60% w/w by weight of the hydrogel particle. In other embodiments, the carrier is present in an amount of from about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, or 59%, to about 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% w/w by weight of the hydrogel particle.

Regarding the protein involved in the method of encapsulation, in some embodiments, the protein comprises at least one of a chickpea protein, a lentil protein, a faba bean protein, a soybean protein, or combinations thereof. In some preferred embodiments, the protein is a faba bean protein while in some particularly preferred embodiments, the faba bean protein is unhydrolyzed. These inventors have found that unhydrolyzed protein, sometimes described as native protein, has sufficient in-tact protein to form a hydrogel. In proteins that have been hydrolyzed, the hydrolysis process can limit the ability of the protein to form a hydrogel. Faba beans (Vicia faba), used interchangeably with fava beans, provide a good source of sustainable plant protein and are currently underutilized as an ingredient in the encapsulation industry. In some embodiments, the protein has a protein content of at least 85% w/w by weight of the protein. In some preferred embodiments, the protein is a faba bean protein isolate with a protein content of at least 85% w/w by weight of the protein. In some preferred embodiments, the protein is an unhydrolyzed faba bean protein isolate with a protein content of at least 85% w/w by weight of the protein.

In some embodiments, the protein is present in the hydrogel an amount of at least 7% w/v by weight of the hydrogel. In other embodiments, the protein is present in the hydrogel in amount of at least 8% w/v, at least 9% w/v, at least 10% w/v, at least 11% w/v, at least 12% w/v, at least 13%, w/v, at least 14% w/v, or at least 15% w/v, all by weight of the hydrogel. In still other embodiments, the protein is present in the hydrogel in an amount of from about 7% w/v to about 20% w/v by weight of the hydrogel. In some embodiments, the protein is present in the hydrogel in an amount of from about 7% w/v, 8% w/v, 9% w/v, 10% w/v, 11% w/v, 12% w/v, 13% w/v, 14% w/v, 15% w/v, 16% w/v, 17% w/v, 18% w/v, or 19% w/v to about 8% w/v, 9% w/v, 10% w/v, 11% w/v, 12% w/v, 13% w/v, 14% w/v, 15% w/v, 16% w/v, 17% w/v, 18% w/v, 19% w/v, or 20% w/v all by weight of the hydrogel.

In embodiments where the hydrogel undergoes a drying step to form a hydrogel particle, the protein is present in the hydrogel particle in an amount of from about 10% w/w to about 50% w/w. In some embodiments, the protein is present in an amount of from about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49% to about 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w by weight of the hydrogel particle.

In some embodiments, gelling agents, cross-linking agents or combinations thereof may be mixed with the emulsified active and heated to a temperature of about 25 C to 60 C prior to the addition of the protein. Suitable gelling agents can include starches (such as gelling starches or native starches), hydrocolloids, pectin, alginates, gums, fibers or cellulosic compounds. Suitable cross-linking agents can include enzymes, glutaraldehyde, acetaldehyde, or salts such as calcium salts (e.g., calcium chloride), magnesium salts (e.g., magnesium phosphate), or zinc salts (e.g., zinc sulphate).

In some embodiments, the method of encapsulation further comprises mixing the active with a weighting agent prior to the homogenization step. In some of these embodiments, the weighting agent comprises at least one of a sucrose acetate isobutyrate, an ester gum, or combinations thereof. In other of these embodiments, it is advantageous for the active to be a flavor, a fragrance, a pro-fragrance, or any other active with a specific gravity of less than one. By combining the active with the weighting agent, stability of the hydrogel can be improved.

In some embodiments, the homogenization step of the method of encapsulation further comprises an antioxidant. In these embodiments, the antioxidant can include, but is not limited to, carotenoids (examples include beta-carotene, lycopene, lutein, zeaxanthin); vitamins (examples include vitamin C (Ascorbic Acid) or an ester thereof, vitamin A or an ester thereof, vitamin E (alpha-tocopherol) or an ester thereof; vitamin-like antioxidants such as coenzyme Q10 (CoQ10) and glutathione; plant extracts such as oregano, lemon balm or rosemary extract; glucosinolate derivatives (compounds in this group include isothiocyanates, thiocyanates, indoles, nitrils); polyphenolics (chlorogenic acid found in coffee, resveratrol in red wine and flavonoids fall into this chemical group); lignan, selenium, flavonoids, BHA (butylated hydroxyanisole), BHT (butylated hydroxytoluene), TBHQ (tert-butyl hydroquinone), propyl gallate, and antioxidant enzymes such as superoxide dismutase (SOD), catalase, and glutathione peroxidase. In some embodiments, the antioxidant includes at least one of a carotenoid, a vitamin, BHA, BHT, TBHQ, propyl gallate, a polyphenolic, a glucosinolate, rosemary extract or combinations thereof. In some embodiments, the antioxidant used in the method of encapsulation increases the hydrogel stability by protecting the active from oxidation.

In some embodiments, the mixing step further comprises an acidulant. Those of skill in the art will be familiar with convention acidulants. Non-limiting examples of acidulants can include, but are not limited to, citric acid, malic acid, acetic acid, phosphoric acid, tartaric acid, sulfuric acid, glucono delta lactone, and the like. The acidulant can serve to lower the pH of the composition to near the isoelectric point of the protein which can reduce charge repulsion and promote protein to protein interactions which enhances the efficacy of the hydrogel. In some embodiments, the pH of the mixture is from about 4.5 to about 5.5.

In some embodiments, these inventors have developed a method of improving the heat stability of an active wherein the method comprises the steps of homogenizing an emulsifier with the active and a carrier to create an emulsified active, heating the emulsified active to a first temperature of at least 35 C, mixing the heated emulsified active with a protein, and heating the mixture to a second temperature of at least 50 C to create a hydrogel; wherein the active exhibits heat stability. As used herein, the term “heat stability” refers to any property of the hydrogel that demonstrates a protective effect on the active. In some embodiments, heat stability is measured by at least one of volatile retention, time intensity, or combinations thereof. In some embodiments, volatile retention is determined by gas chromatography (GC/FID using Agilent 6850 or equivalent) and is expressed as a percentage of the initial amount of flavor in the flavored hydrogel particle. In some embodiments, time intensity is measured by sensory testing where panelists rate the intensity of all the attributes listed for each sample using Labeled Magnitude Scale (LMS) rating from 0 (no sensation) to 100 (strongest ever experienced). The time-intensity data are collected using the Compusense's continuous time-intensity measurement program.

In some embodiments, the method further comprises a step of holding the mixture at the second temperature for a gelation time of at least 30 minutes. In other embodiments, the method further comprises a step of cooling the hydrogel to a cooling temperature of from about 50 C to about 70 C. And in still other embodiments, the method further comprises a step of drying the hydrogel to create hydrogel particles.

In some preferred embodiments, the method of encapsulation provides a flavored hydrogel particle. In these embodiments, the method of encapsulation comprises the steps of dissolving an emulsifier and a carrier in a quantity of water to form an aqueous matrix. The aqueous matrix is then homogenized with a flavor to form a flavor emulsion. Next, the flavor emulsion is heated to a first temperature of at least 50 C and a protein is mixed in. The mixture is then heated to a second temperature of at least 70 C and held at the second temperature for a gelation time of at least 30 minutes to create a hydrogel. To complete the method, the hydrogel is cooled to a temperature of from about 50 C to 70 C and dried to generate hydrogel particles.

In some of these embodiments, the hydrogel particles have a mean particle size of from about 120 microns to about 130 microns. In other embodiments, the hydrogel particles have a mean particle size of from about 120, 121, 122, 123, 124, 125, 126, 127, 128, or 129 microns to about 121, 122, 123, 124, 125, 126, 127, 128, 129, or 130 microns.

Additionally, in some of these embodiments, the hydrogel particles have a volatile retention of at least 80% as measured by gas chromatography (GC/FID using Agilent 6850 or equivalent) and as expressed as a percentage of the initial amount of flavor in the flavored hydrogel particle. In other embodiments, the hydrogel particles have a volatile retention of at least 90% as measured by the same method.

In some embodiments, the hydrogel particle contains an amount of flavor of from about 0.1% to about 60% w/w by weight of the hydrogel particle. In some embodiments, the hydrogel particle contains an amount of flavor of from about 0.1% to about 5% w/w by weight of the hydrogel particle, in other embodiments, the hydrogel particle contains an amount of flavor of from about 0.25% to about 7% w/w by weight of the hydrogel particle, and, in still other embodiments, the hydrogel particle contains an amount of flavor of from about 5% to about 15% w/w by weight of the hydrogel particle. In some embodiments, the amount of flavor in the hydrogel particle is from about 10% to about 30% w/w by weight of the hydrogel particle, and in other embodiments, the amount of flavor in the hydrogel particle is from about 15% to about 40% w/w by weight of the hydrogel particle, and in other embodiments, the amount of flavor is from about 20% to about 50% w/w by weight of the hydrogel particle, while in still other embodiments, the amount of flavor is from about 25% to about 60% w/w by weight of the hydrogel particle.

In some embodiments, the flavor comprises at least one of fruit flavor, a savory flavor, a dairy flavor, a bakery flavor, a reaction flavor, a taste modifier, a sweetness modifier, a cooling agent, a warming agent, a flavor enhancer, or combinations thereof.

In some embodiments, the method of encapsulation further comprises dissolving a weighting agent in the flavor prior to the homogenization step. In some embodiments, the weighting agent comprises at least one of a sucrose acetate isobutyrate, an ester gum, or combinations thereof.

In addition to methods of encapsulation, these inventors have developed microcapsules (also referred to as encapsulations or encapsulation systems) and products containing the microcapsules. In some embodiments, the encapsulation is the material created by performing the steps in the method of encapsulation including the steps of homogenizing an emulsifier with an active and a carrier to create an emulsified active, heating the emulsified active to a first temperature of at least 35 C followed by the steps of mixing the heated emulsified active with a protein and heating the mixture to a second temperature of at least 50 C to create a hydrogel. Additional steps can include holding the hydrogel at the second temperature for a gelation time of at least 30 minutes and then cooling and drying the encapsulation to obtain a hydrogel particle.

In some embodiments, a microcapsule comprises an active and a shell wherein the active comprises a flavor and the shell comprises a faba bean protein and the microcapsule is characterized by having a volatile retention of at least 80%. In other embodiments, the microcapsule has a volatile retention of at least 90%. In other embodiments, a microcapsule comprises an active and a shell obtainable by the method steps described herein wherein the active comprises a flavor and the shell comprises a faba bean protein and the microcapsule is characterized by having a volatile retention of at least 80%. In other embodiments, the microcapsule has a volatile retention of at least 90%. Volatile retention can be measured using the gas chromatographic method described in Example 4. In some of these embodiments, the microcapsule includes dried hydrogel particles and the flavor in the microcapsule is present in an amount of from about 0.1% to about 60% w/w by weight of the microcapsule.

In some embodiments, the flavor in the microcapsule can include one or more volatile and nonvolatile compounds. A variety of flavors can be used in accordance with the present invention. Flavors may be chosen from synthetic flavors, flavoring oils and oil extracts derived from plants, leaves, flowers, fruits, and combinations thereof. Representative flavor oils include, but are not limited to, spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit, and fruit essences including apple, pear, peach, strawberry, watermelon, raspberry, cherry, plum, pineapple, apricot and so forth. These flavors can be used individually or in an admixture.

Volatile compounds in the flavor oils may include, but are not limited to, acetaldehyde, dimethyl sulfide, ethyl acetate, ethyl propionate, methyl butyrate, and ethyl butyrate. Flavors containing volatile aldehydes or esters include, e.g., cinnamyl acetate, cinnamaldehyde, citral, diethylacetal, dihydrocarvyl acetate, eugenyl formate, and p-methylanisole. Further examples of volatile compounds that may be present in the flavor oils include acetaldehyde (apple); benzaldehyde (cherry, almond); cinnamic aldehyde (cinnamon); citral, i.e., alpha citral (lemon, lime); neral, i.e., beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e., trans-2 (berry fruits); tolyl aldehyde (cherry, almond); veratraldehyde (vanilla); 2,6-dimethyl-5-heptenal, i.e., melonal (melon); 2-6-dimethyloctanal (green fruit); and 2-dodecenal (citrus, mandarin); cherry; or grape and mixtures thereof. The hydrogel composition may also contain taste modulators and artificial sweeteners.

The flavor can also contain the following:
(i) taste masking agents, substances for masking one or more unpleasant taste sensations, in particular a bitter, astringent and/or metallic taste sensation or aftertaste. Examples include lactisol [2O-(4-methoxyphenyl) lactic acid] (cf. U.S. Pat. No. 5,045,336), 2,4-dihydroxybenzoic acid potassium salt (cf. U.S. Pat. No. 5,643,941), ginger extracts (cf. GB 2,380,936), neohesperidine dihydrochalcone (cf. Manufacturing Chemist 2000, July issue, p. 16-17), specific flavones (2-phenylchrom-2-en-4-ones) (cf. U.S. Pat. No. 5,580,545), specific nucleotides, for example cytidine-5′-monophosphates (CMP) (cf. US 2002/0177576), specific sodium salts, such as sodium chloride, sodium citrate, sodium acetate and sodium lactate (cf. Nature, 1997, Vol. 387, p. 563), a lipoprotein of .beta.-lactoglobulin and phosphatidic acid (cf. EPA 635 218), neodiosmine [5,7-dihydroxy-2-(4-methoxy-3-hydroxyphenyl)-7-O-neohesperidosyl-chrom-2-en-4-one] (cf. U.S. Pat. No. 4,154,862), preferably hydroxyflavanones according to EP 1 258 200, in turn preferred in this respect 2-(4-hydroxyphenyl)-5,7-dihydroxychroman-4-one (naringenin), 2-(3,4-dihydroxyphenyl)-5,7-dihydroxychroman-4-one (eriodictyol), 2-(3,4-dihydroxyphenyl)-5-hydroxy-7-methoxychroman-4-one (eriodictyol-7-methylether), 2-(3,4-dihydroxyphenyl)-7-hydroxy-5-methoxychroman-4-one (eriodictyol-5-methylether) and 2-(4-hydroxy-3-methoxyphenyl)-5,7-dihydroxychroman-4-one (homoeriodictyol), the (2S)- or (2R)-enantiomers thereof or mixtures thereof as well as the mono- or polyvalent phenolate salts thereof with Na+, K+, NH4+, Ca2+, Mg2+ or Al3+as counter cations or .gamma.-aminobutyric acid (4-aminobutyric acid, as the neutral form (“inner salt”) or in the carboxylate or ammonium form) according to WO 2005/09684;
(ii) taste sensates including hot tasting, salivation-inducing substances, substances causing a warmth or tingling feeling, and cooling active ingredients. Examples of hot tasting and/or salivation-inducing substances and/or substances which cause a feeling of warmth and/or a tingling feeling on the skin or on the mucous membranes and which can be a constituent of the products according to the invention are: capsaicin, dihydrocapsaicin, gingerol, paradol, shogaol, piperine, carboxylic acid-N-vanillylamides, in particular nonanoic acid-N-vanillylamide, pellitorin or spilanthol, 2-nonanoic acid amides, in particular 2-nonanoic acid-N-isobutylamide, 2-nonanoic acid-N-4-hydroxy-3-methoxyphenylamide, alkyl ethers of 4-hydroxy-3-methoxybenzyl alcohol, in particular 4-hydroxy-3-methoxybenzyl-n-butylether, alkyl ethers of 4-acyloxy-3-methoxybenzyl alcohol, in particular 4-acetyloxy-3-methoxybenzyl-n-butylether and 4-acetyloxy-3-methoxybenzyl-n-hexylether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylene dioxybenzyl alcohol, (4-hydroxy-3-methoxyphenyl)acetic acid amides, in particular (4-hydroxy-3-methoxyphenyl)acetic acid-N-n-octylamide, vanillomandelic acid alkylamides, ferulic acid-phenethylamides, nicotinaldehyde, methylnicotinate, propylnicotinate, 2-butoxyethylnicotinate, benzylnicotinate, 1-acetoxychavicol, polygodial and isodrimeninol, further preferred cis- and/or trans-pellitorin according to WO 2004/000787 or WO 2004/043906, alkenecarboxylic acid-N-alkylamides according to WO 2005/044778, mandelic acid alkylamides according to WO 03/106404 or alkyloxyalkanoic acid amides according to WO 2006/003210. Examples of preferred hot tasting natural extracts and/or natural extracts which cause a feeling of warmth and/or a tingling feeling on the skin or on the mucous membranes and which can be a constituent of the products according to the invention are: extracts of paprika, extracts of pepper (for example capsicum extract), extracts of chili pepper, extracts of ginger roots, extracts of Aframomum melgueta, extracts of Spilanthes-acmella, extracts of Kaempferia galangal or extracts of Alpinia galanga. Suitable cooling active ingredients include the following: 1-menthol, d-menthol, racemic menthol, menthone glycerol acetal (trade name: Frescolat® MGA), menthyl lactate (trade name: Frescolat® ML, menthyl lactate preferably being 1-menthyl lactate, in particular 1-menthyl-1-lactate), substituted menthyl-3-carboxamides (for example menthyl-3-carboxylic acid-N-ethylamide), 2-isopropyl-N-2,3-trimethyl-butanamide, substituted cyclohexane carboxamides, 3-menthoxypropane-1,2-diol, 2-hydroxyethyl menthyl carbonate, 2-hydroxypropyl menthyl carbonate, N-acetylglycine menthyl ester, isopulegol, hydroxycarboxylic acid menthyl esters (for example menthyl-3-hydroxybutyrate), monomenthyl succinate, 2-mercaptocyclodecanone, menthyl-2-pyrrolidin-5-onecarboxylate, 2,3-dihydroxy-p-menthane, 3,3,5-trimethylcyclohexanone glycerol ketal, 3-menthyl-3,6-di- and -trioxaalkanoates, 3-menthyl methoxyacetate and icilin. Cooling active ingredients which are particularly preferred are as follows: 1-menthol, racemic menthol, menthone glycerol acetal (trade name: Frescolat® MGA), menthyl lactate (preferably 1-menthyl lactate, in particular 1-menthyl-1-lactate, trade name: Frescolat® ML), 3-menthoxypropane-1,2-diol, 2-hydroxyethyl menthyl carbonate, 2-hydroxypropyl menthyl carbonate.

In addition to inventive methods, encapsulations, and microcapsules, these inventors have also developed inventive products comprising a product base and a microcapsule wherein the microcapsule comprises an active and a shell wherein the shell comprises a faba bean protein and wherein the microcapsule has a volatile retention of at least 80%. In some embodiments, the product comprises a microcapsule having a volatile retention of at least 90%.

In some embodiments, the active comprises at least one of a fragrance, a pro-fragrance, a flavor, a malodor counteractive agent, a vitamin, a vitamin derivative, an anti-inflammatory agent, a fungicide, an anesthetic, an analgesic, an antimicrobial active, an anti-viral agent, an anti-infectious agent, a pharmaceutical agent, a nutraceutical agent, an anti-acne agent, a skin lightening agent, an insect repellant, an animal repellent, a vermin repellent, an emollient, a skin moisturizing agent, a wrinkle control agent, a UV protection agent, a fabric softener active, a hard surface cleaning active, a skin conditioning agent, a hair conditioning agent, a flame retardant, an antistatic agent, a taste modulator, a cell, a probiotic, a colorant, a vegetable oil, a fish oil, or combinations thereof.

In some product embodiments wherein the active comprises a flavor, the flavor comprises at least one of a fruit flavor, a savory flavor, a dairy flavor, a bakery flavor, a reaction flavor, a taste modifier, a sweetness modifier, a cooling agent, a warming agent, a flavor enhancer, or combinations thereof. In some of these flavor-containing product embodiments, the product has a higher flavor intensity score as measured by sensory testing than an isoflavored product without the microcapsule.

In other embodiments, the product includes a flavor-containing microcapsule wherein the flavor is present in the microcapsule in an amount of from about 0.1% to about 60% w/w by weight of the microcapsule. In some embodiments, the microcapsule contains an amount of flavor of from about 0.1% to about 5% w/w by weight of the microcapsule, in other embodiments, the microcapsule contains an amount of flavor of from about 0.25% to about 7% w/w by weight of the microcapsule, and, in still other embodiments, the microcapsule contains an amount of flavor of from about 5% to about 15% w/w by weight of the microcapsule. In some embodiments, the amount of flavor in the microcapsule is from about 10% to about 30% w/w by weight of the microcapsule, and in other embodiments, the amount of flavor in the microcapsule is from about 15% to about 40% w/w by weight of the microcapsule, and in other embodiments, the amount of flavor is from about 20% to about 50% w/w by weight of the microcapsule, while in still other embodiments, the amount of flavor is from about 25% to about 60% w/w by weight of the microcapsule.

In some embodiments, the microcapsule is present in the product in an amount of from about 0.1% to about 10% w/w by weight of the product. In some embodiments, the microcapsule is present in the product in an amount of from about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, or 9.5% to about 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% w/w by weight of the product.

In some embodiments, the product base comprises at least one of a food product base, a pharmaceutical product base, a cosmetic product base, a consumer product base, or combinations thereof. In some particular food product base embodiments, the food product base comprises at least one of a chewing gum, a confection, a beverage, a snack, a dairy product, a soup, a sauce, a condiment, a cereal, a baked good, meat products or combinations thereof. In other food product base embodiments, the food product base comprises a pet food, pet treat or pet oral care product.

In some particular consumer product base embodiments, the consumer product base comprises at least one of an oral care product, a detergent, a fabric softener, a fabric care product, an antiperspirant, a deodorant, a talcum powder, a kitty litter, a hair care product, a styling product, a personal care product, an air freshener, a cleaner, or combinations thereof.

The following are provided as specific embodiments of the present invention. Other modifications of this invention will be readily apparent to those skilled in the art. Such modifications are understood to be within the scope of these inventions. As used herein, all percentages are weight percent unless otherwise noted, ppm is understood to stand for parts per million, L or 1 is understood to be liter, ml is understood to be milliliter, g is understood to be gram, Kg is understood to be kilogram, mol is understood to be mole, mmol is understood to be millimole, psig is understood to be pound-force per square inch gauge, and mmHg be millimeters (mm) of mercury (Hg).

### EXAMPLES

### Examples 1-6. Testing Methods

### Example 1—Apparent Viscosity

The apparent viscosity of feed slurries was measured using Brookfield viscometer using spindles #4 at 60 RPM.

### Example 2—Droplet and Particle Size Measurements

The droplet size distribution of the flavors in the feed slurry was measured using a Beckman Coulter LS 13 320 Laser Diffraction Particle Size Analyzer. Distilled water was used as the dispersant.

The powder particle size distribution of the dried microcapsules was measured using a Mastersizer 3000 laser light scattering instrument (Malvern Instruments Ltd., Worcestershire, United Kingdom) equipped with a powder sample handling unit.

Droplet/particle distributions were calculated by instrument according to the Fraunhofer Theory.

Droplet/particle size measurements were reported as volume-surface mean diameters or D.

### Example 3—Microcapsule Surface and Total Oil Measurements

A1.0-2.0 g sample was weighed into a 4-dram vial, and the exact weight was recorded. Then 6.7 g (approx. 10 ml) of hexanes were added, and the exact weight was recorded. The sample was placed on the tube rotator for 10 minutes. An aliquot with a 0.45 μm nylon syringe filter was then filtered into an autosampler vial. The surface oil was measured using a gas chromatographic (GC) instrument equipped with flame ionization detector and quantified using standard solutions. Surface oil represents the portion of oil present on the surface of the microcapsule (Bao et al. 2011). Total oil content of the microcapsules was determined based on calculation of total volatile retention which has been described below. Encapsulation efficiency was calculated as follows: (total oil content−surface oil)/total oil content×100%

### Example 4—Flavor Volatile Retention Measurements

The amounts of individual aroma compounds retained in flavor encapsulated powders were determined by gas chromatography (GC). Approximately 1.0 g of spray dry powder or 3 g of ground crackers were weighed into a 50 ml centrifuge tube, then, 5 ml of deionized water was added into the tube, and vortexed at high speed for 1 min to dissolve or homogenize the sample. When the sample was totally dissolved, 5 ml of extraction solvent (acetonitrile containing 0.05% ethyl valerate as internal standard) was pipetted into the same tube, and vortexed at high speed for 1 min. Then 4.0 g anhydrous magnesium sulfate was added into the sample mixture and vortexed immediately at high speed for 1 min. The tubes were then centrifuged, and clear extract was transferred to a 2 ml screw-cap vial and analyzed by GC/FID (Agilent 6850 or equivalent). Concentrations of individual aroma compounds (mg/g powder) in each spray dried powder were determined via linear regression (R2>0.99) obtained from 3-point calibration curves using neat oil, which went through the same extraction procedure. Target flavor components were identified using both retention indices and/or mass spectrometry. The aroma retentions of the individual compounds were expressed as a percentage of their initial amount in the flavor powders.

### Example 5—Cracker Preparation Method

The flavored crackers were made by adding oil and water to dry ingredients per the below formula to make a dough. The dough was then proofed for 2 hours followed by sheeting and cutting the dough to form small crackers. The crackers were baked at 380 F for 6 min and 230 F for 4 min.

TABLE 1
Cracker Composition

| Ingredient | % |
|---|---|
| Water | 22.2 |
| Bread Flour | 63.3 |
| Sugar | 4.37 |
| Baking soda | 0.59 |
| Salt | 0.55 |
| Vegetable Oil | 6.71 |
| Ammonium Bicarbonate | 1.78 |
| Spray Dried Flavor System | 0.4 (approximately |
| | 0.1% flavor oil) |

### Example 6—Sensory Evaluation Method

A group of trained panelists participated in sensory evaluation of the flavored crackers. Panelists were presented with new blind coded samples. Each panelist rated intensity of all the attributes listed for each sample using Labeled Magnitude Scale (LMS) rating from 0 (no sensation) to 100 (strongest ever experienced). The time-intensity data were collected using the Compusense's continuous time-intensity measurement program. Panelists tasted a piece of cracker and continuously measured the flavor intensity (butter, tomato, strawberry, etc.) for a total of 2 min. They used water to clean their mouths thoroughly and took a minimum 2 min break before evaluating their next samples. The evaluation order of samples was balanced among panelists by Williams Latin Square design. There were five replications/sets for cracker evaluation.

### Examples 7-12 Hydrogel Encapsulated Flavors and Methods of Making Same

### Example 7. Method of Encapsulating Butter Flavor

TABLE 2
Encapsulated Butter Flavor Composition Prior to Spray Drying

| Materials | Parts | % |
|---|---|---|
| Water | 5320 | 46.27 |
| Emulsifier - gum arabic with trade name Gum | 1320 | 11.47 |
| Eficacia ™ from Nexira | | |
| Carrier - spray dried glucose syrup with trade | 1500 | 13.04 |
| name Glucidex 47 ™ from Roquette | | |
| Protein - Faba bean protein with >85% protein | 1400 | 12.17 |
| content from AGT foods | | |
| Acidulant - Glucono-delta-lactone from | 100 | 0.88 |
| Triconor Distribution | | |
| Flavor - Butter Flavor from IFF | 1500 | 13.04 |
| Weighting agent - Sucrose Acetate | 360 | 3.13 |
| Isobutyrate (SAIB) from Eastman | | |
| Total | 11,500 | 100 |

### Process:

1. The emulsifier and carrier were dissolved in ambient temperature water using an overhead mixer by stirring at 500 rpm for at least one hour until the solids were fully dissolved.
2. In a separate container, the SAIB was dissolved in the butter flavor.
3. The oil phase was mixed with the emulsifier/carrier solution and homogenized using a Silverson Verso in-line rotor-stator mixer operating at 2500 or 3450 rpm and a circulation rate of 1 kilogram per minute.
4. The homogenized slurry was then heated to about 60 C followed by addition of the acidulant and protein with mixing until both the acidulant and protein were fully dissolved.
5. The protein-containing slurry was then heated to 75-80 C and held for at least 30 minutes with continuous mixing.
6. The slurry was then cooled to 50-70 C prior to spray drying using a pilot-scale Anhydro MicraSpray MS-400 spray dryer connected to a fluid bed unit. The inlet air temperature was adjusted to around 95 C, and the outlet temperature was kept at around 55 C by controlling the flow rate. A two-fluid nozzle atomizer was used to atomize the aqueous flavor slurry inside the dryer at feed rate of 10 kg/h and air flow rate of 200 cubic feet/min. In order to maintain homogeneity, the slurry was gently stirred while fed into the spray dryer.
7. The finished flavor encapsulated powder was collected in the cyclone collection vessel and stored in sealed aluminum bag at −5 C until analysis.

### Experimental Results:

The hydrogel liquid flavor emulsion prior to spray drying showed flavor droplets with mean size of 2.06 μm and viscosity of around 956 Cps at 40 C.

The spray dried butter-flavored hydrogel particles had mean particle size diameter of around 128 μm and volatile retention of 98% with trace amount of free oil indicating strong encapsulation efficiency.

The performance of butter hydrogel particles was evaluated in cracker as a model of baked products. The cracker was made as shown in Example 5. The sensory performance of hydrogel particles was assessed against benchmark (i.e. not hydrogel) spray dried flavor at the same active level of flavor oil (i.e. iso-active flavor level) in the cracker. Table 3 shows the trained panel sensory results and Table 4 shows measured volatile retention in crackers. Both cracker samples contained as iso-active butter flavor level of 0.1% neat oil equivalent. Both data sets show that the inventive butter hydrogel particle outperformed the spray dried comparative benchmark sample by having significantly higher flavor intensity in sensory testing and higher retained actives in cracker as measured by chromatography technique. The results illustrated that encapsulating flavoring compounds in hydrogel structure enhanced the active protection during dough preparation and baking conditions resulting in a finished product with enhanced sensory performance.

TABLE 3
Time Intensity Sensory Results for Cracker Samples

| | | Hydrogel |
|---|---|---|
| | Benchmark | Particle - |
| | sample - cracker | cracker with |
| | with spray dried | hydrogel particle |
| Time | butter flavor | from Example 7 |
| (Seconds) | (LMS) | (LMS) |
| 0 | 0.0 | 0.0 |
| 5 | 0.5 | 0.4 |
| 10 | 1.4 | 1.1 |
| 15 | 2.7 | 2.7 |
| 20 | 4.0 | 4.6 |
| 25 | 5.2 | 6.4 |
| 30 | 6.0 | 7.4 |
| 35 | 6.9 | 8.3 |
| 40 | 7.5 | 9.0 |
| 45 | 7.8 | 9.5 |
| 50 | 8.1 | 9.6 |
| 55 | 8.0 | 9.7 |
| 60 | 7.8 | 9.7 |
| 65 | 7.6 | 9.4 |
| 70 | 7.4 | 8.9 |
| 75 | 7.3 | 8.7 |
| 80 | 7.0 | 8.4 |
| 85 | 6.7 | 8.1 |
| 90 | 6.4 | 8.0 |
| 95 | 6.0 | 7.6 |
| 100 | 5.8 | 7.2 |
| 105 | 5.6 | 6.8 |
| 110 | 5.3 | 6.5 |
| 115 | 4.9 | 6.2 |
| 120 | 4.7 | 5.9 |

TABLE 4
Volatile Retention Results for Cracker Samples
(expressed at % of initial amount of the compound as measured by GC)

| | | decalactone, | decalactone, | |
|---|---|---|---|---|
| | Acetoin | gamma | delta | Total |
| Benchmark sample | 12.4% | 34.3% | 13.7% | 19.7% |
| (cracker with spray dried | | | | |
| butter flavor) | | | | |
| Hydrogel Particle | 13.3% | 45.2% | 16.9% | 24.2% |
| (cracker with hydrogel | | | | |
| particle from Example 7) | | | | |

### Example 8. Method of Encapsulating Tomato Flavor

TABLE 5
Encapsulated Tomato Flavor Composition Prior to Spray Drying

| Materials | Parts | % |
|---|---|---|
| Water | 5320 | 46.27 |
| Emulsifier - gum arabic with trade | 1320 | 11.47 |
| name Gum Eficacia ™ from Nexira | | |
| Carrier - spray dried glucose syrup | 1500 | 13.04 |
| with trade name Glucidex 47 ™ | | |
| from Roquette | | |
| Protein - Faba bean protein from | 1400 | 12.17 |
| AGT foods | | |
| Acidulant - Glucono-delta-lactone | 100 | 0.88 |
| from Triconor Distribution | | |
| Flavor - Tomato Flavor from IFF | 1500 | 13.04 |
| Weighting agent - Sucrose Acetate | 360 | 3.13 |
| Isobutyrate (SAIB) from Eastman | | |
| Total | 11,500 | 100 |

### Process: Same as in Example 7.

### Experimental Results:

The tomato slurry before spray drying showed droplets with mean size of 1.46 μm and viscosity of around 1350 Cps at 40 C.

The dry tomato hydrogel particles had mean particle size diameter of around 126 μm and volatile retention of 74.8% with trace amount of free oil.

The sensory performance of tomato hydrogel particles was evaluated in cracker. The tomato flavored crackers were made as shown in Example 5 containing 0.1% flavor neat oil equivalent.

The flavored crackers were then subjected to sensory evaluation by trained panelists and results were summarized in Table 6. Both cracker samples contained as iso-active tomato flavor level of 0.1% neat oil equivalent. As shown, tomato crackers made with the inventive hydrogel particles outperformed crackers containing the benchmark spray dried sample indicating that the hydrogel particles provide better flavor protection during dough preparation and baking steps compared to benchmark technology resulting in enhanced sensory performance. Due to extremely low levels of active compounds in baked tomato crackers, the amount of retained actives was not detectable using chromatography technique.

TABLE 6
Time Intensity Sensory Results for Cracker Samples

| | Benchmark sample - | Hydrogel Particle - |
|---|---|---|
| | cracker with | cracker with |
| | spray dried tomato | hydrogel particle |
| Time | flavor | from Example 8 |
| (Seconds) | (LMS) | (LMS) |
| 0 | 0.0 | 0.0 |
| 5 | 0.7 | 0.7 |
| 10 | 1.6 | 1.8 |
| 15 | 2.5 | 3.1 |
| 20 | 3.7 | 4.5 |
| 25 | 4.9 | 5.6 |
| 30 | 5.7 | 6.4 |
| 35 | 6.3 | 7.2 |
| 40 | 6.9 | 7.7 |
| 45 | 7.1 | 7.8 |
| 50 | 7.1 | 8.0 |
| 55 | 7.1 | 8.0 |
| 60 | 6.9 | 7.9 |
| 65 | 6.6 | 7.5 |
| 70 | 6.3 | 7.1 |
| 75 | 5.7 | 6.6 |
| 80 | 5.4 | 6.1 |
| 85 | 5.1 | 5.8 |
| 90 | 4.8 | 5.6 |
| 95 | 4.6 | 5.2 |
| 100 | 4.3 | 4.7 |
| 105 | 4.1 | 4.4 |
| 110 | 3.9 | 4.2 |
| 115 | 3.5 | 3.9 |
| 120 | 3.2 | 3.5 |

### Example 9. Method of Encapsulating Butter Flavor with No Weighting Agent

TABLE 7
Encapsulated Butter Flavor Composition Prior to Spray Drying

| Materials | Parts | % |
|---|---|---|
| Water | 5320 | 46.27 |
| Emulsifier - gum arabic with trade | 1320 | 11.47 |
| name Gum Eficacia ™ from Nexira | | |
| Carrier - spray dried glucose syrup | 1860 | 16.17 |
| with trade name Glucidex 47 ™ | | |
| from Roquette | | |
| Protein - Faba bean protein from | 1400 | 12.17 |
| AGT foods | | |
| Acidulant - Glucono-delta-lactone | 100 | 0.88 |
| from Triconor Distribution | | |
| Flavor - Butter Flavor from IFF | 1500 | 13.04 |
| Total | 11,500 | 100 |

### Process: Same as in Example 7.

### Experimental Results:

In the absence of sucrose acetate isobutyrate as a weighting agent, the flavor slurry showed flavor droplets with mean size of around 2 μm and viscosity of around 1037 Cps at 40 C.

The dry hydrogel particles had a mean particle size of around 89.9 μm and volatile retention of 100.0% with trace amount of surface oil. The volatile retention results indicated very strong encapsulation efficiency even in the absence of sucrose acetate isobutyrate.

The crackers made with this sample using the composition and method in Example 5 revealed significantly better performance compared to benchmark sample as reported by trained sensory panelists (Table 8) and retained volatiles in the cracker as measured by GC (Table 9). Both cracker samples contained as iso-active butter flavor level of 0.1% neat oil equivalent. These results illustrate the robustness of hydrogel particles in protecting active compounds during dough preparation and baking conditions.

TABLE 8
Time Intensity Sensory Results for Cracker Samples

| | Benchmark sample - | Hydrogel Particle - |
|---|---|---|
| | cracker with spray dried | cracker with hydrogel |
| Time | butter flavor | particle from Example 9 |
| (Seconds) | (LMS) | (LMS) |
| 0 | 0.0 | 0.0 |
| 5 | 0.4 | 0.6 |
| 10 | 1.4 | 1.7 |
| 15 | 2.5 | 3.4 |
| 20 | 3.9 | 5.4 |
| 25 | 5.0 | 6.9 |
| 30 | 6.1 | 7.7 |
| 35 | 7.1 | 8.4 |
| 40 | 7.7 | 9.0 |
| 45 | 8.0 | 9.2 |
| 50 | 8.3 | 9.4 |
| 55 | 8.4 | 9.4 |
| 60 | 8.2 | 9.3 |
| 65 | 7.8 | 9.0 |
| 70 | 7.6 | 8.5 |
| 75 | 7.2 | 8.2 |
| 80 | 6.9 | 7.9 |
| 85 | 6.5 | 7.5 |
| 90 | 6.1 | 7.2 |
| 95 | 5.6 | 6.8 |
| 100 | 5.3 | 6.4 |
| 105 | 5.1 | 5.8 |
| 110 | 4.7 | 5.5 |
| 115 | 4.5 | 5.3 |
| 120 | 4.2 | 5.2 |

TABLE 9
Volatile Retention Results for Cracker Samples (expressed
at % of initial amount of the compound as measured by GC)

| | | decalactone, | decalactone, | |
|---|---|---|---|---|
| | Acetoin | gamma | delta | Total |
| Benchmark sample | 13.0% | 50.1% | 25.1% | 26.6% |
| (cracker with spray dried | | | | |
| butter flavor) | | | | |
| Hydrogel Particle | 14.7% | 63.3% | 30.0% | 32.5% |
| (cracker with hydrogel | | | | |
| particle from Example 9) | | | | |

### Example 10. Method of Encapsulating Tomato Flavor with No Weighting Agent

TABLE 10
Encapsulated Tomato Flavor Composition Prior to Spray Drying

| Materials | Parts | % |
|---|---|---|
| Water | 5320 | 46.27 |
| Emulsifier - gum arabic with trade | 1320 | 11.47 |
| name Gum Eficacia ™ from Nexira | | |
| Carrier - spray dried glucose syrup | 1860 | 16.17 |
| with trade name Glucidex 47 ™ | | |
| from Roquette | | |
| Protein - Faba bean protein from | 1400 | 12.17 |
| AGT foods | | |
| Acidulant - Glucono-delta-lactone | 100 | 0.88 |
| from Triconor Distribution | | |
| Flavor - Tomato Flavor from IFF | 1500 | 13.04 |
| Total | 11,500 | 100 |

### Process: Same as in Example 7.

### Experimental Results:

In the absence of sucrose acetate isobutyrate (as a weighting agent), the flavor slurry showed flavor droplets with mean size of around 5 μm, which was larger than tomato flavor slurry containing sucrose acetate isobutyrate (Example 8). The viscosity of this slurry was measured at around 1013 Cps at 40 C. The dry hydrogel particles had a mean particle size of around 85.5 μm and volatile retention of 90.1% with trace amount of surface oil. The volatile retention results indicated very strong encapsulation efficiency even in the absence of sucrose acetate isobutyrate. The cracker made with this sample using the composition and method of Example 5 revealed significantly better performance as reported by trained sensory panelists (Table 11).

TABLE 11
Time Intensity Sensory Results for Cracker Samples

| | Benchmark sample - | Hydrogel Particle - |
|---|---|---|
| | cracker with spray dried | cracker with hydrogel |
| Time | tomato flavor | particle from Example 10 |
| (Seconds) | (LMS) | (LMS) |
| 0 | 0.0 | 0.0 |
| 5 | 0.8 | 1.2 |
| 10 | 2.5 | 3.7 |
| 15 | 4.4 | 6.5 |
| 20 | 6.0 | 8.9 |
| 25 | 7.2 | 10.3 |
| 30 | 8.1 | 10.9 |
| 35 | 8.6 | 11.6 |
| 40 | 9.0 | 11.9 |
| 45 | 9.2 | 11.9 |
| 50 | 9.3 | 12.0 |
| 55 | 9.0 | 11.8 |
| 60 | 8.8 | 11.6 |
| 65 | 8.2 | 11.0 |
| 70 | 8.0 | 10.4 |
| 75 | 7.8 | 10.1 |
| 80 | 7.3 | 9.6 |
| 85 | 7.1 | 9.3 |
| 90 | 6.9 | 9.0 |
| 95 | 6.5 | 8.4 |
| 100 | 6.1 | 8.0 |
| 105 | 5.8 | 7.7 |
| 110 | 5.5 | 7.5 |
| 115 | 5.3 | 7.0 |
| 120 | 5.2 | 6.9 |

### Example 11. Method of Encapsulating Strawberry Flavor with No Weighting Agent

TABLE 12
Encapsulated Strawberry Flavor Composition
Prior to Spray Drying

| Materials | Parts |
|---|---|
| Water | 5320 |
| Gum Eficacia | 1320 |
| Glucidex 47 | 1860 |
| Faba bean proteins | 1400 |
| Glucono-delta-lactone | 100 |
| Strawberry Flavor | 1500 |

### Process: Same as in Example 7.

### Experimental Results:

In the absence of sucrose acetate isobutyrate as a weighting agent, the flavor slurry showed flavor droplets with mean size of around 10 μm and viscosity of around 1003 Cps at 40 C.

The dry hydrogel particles had a mean particle size of around 79.1 μm and volatile retention of 93.1% with trace amounts of surface oil. The volatile retention results indicated very strong encapsulation efficiency even in the absence of sucrose acetate isobutyrate.

For the cracker application, strawberry hydrogel particles were dosed in the cracker dough to achieve 0.5% neat oil equivalent according to the composition and method in Example 5. As shown in Table 13, the trained sensory panel reported significantly higher flavor intensity compared to the benchmark sample. To confirm the sensory results, crackers were subjected to volatile retention measurement and results summarized in Table 14. As shown, all three compounds (pineapple compounds, Methyl Cinnamate [Me Cinn], and Gamma Decalactone) measured by GC showed significantly higher concentration than the benchmark sample.

TABLE 13
Time Intensity Sensory Results for Cracker Samples

| | Benchmark sample - | Hydrogel Particle - |
|---|---|---|
| | cracker with spray dried | cracker with hydrogel |
| Time | strawberry flavor | particle from Example 11 |
| (Seconds) | (LMS) | (LMS) |
| 0 | 0.0 | 0.0 |
| 5 | 1.4 | 1.4 |
| 10 | 3.6 | 3.6 |
| 15 | 5.5 | 5.8 |
| 20 | 6.9 | 7.3 |
| 25 | 8.1 | 8.4 |
| 30 | 8.6 | 9.0 |
| 35 | 9.1 | 9.5 |
| 40 | 9.4 | 9.9 |
| 45 | 9.4 | 9.8 |
| 50 | 9.4 | 9.9 |
| 55 | 9.2 | 9.8 |
| 60 | 9.0 | 9.4 |
| 65 | 8.5 | 9.0 |
| 70 | 8.3 | 8.7 |
| 75 | 7.9 | 8.4 |
| 80 | 7.6 | 8.0 |
| 85 | 7.2 | 7.8 |
| 90 | 7.0 | 7.5 |
| 95 | 6.7 | 7.2 |
| 100 | 6.5 | 6.8 |
| 105 | 6.1 | 6.5 |
| 110 | 5.9 | 6.3 |
| 115 | 5.6 | 6.1 |
| 120 | 5.4 | 5.8 |

TABLE 14
Volatile Retention Results for Cracker Samples (expressed
at % of initial amount of the compound as measured by GC)

| | Pineapple | Me | gamma | |
|---|---|---|---|---|
| | compound | Cinn | decalalctone | Total |
| Benchmark sample | 8.7% | 23.7% | 43.1% | 10.4% |
| (cracker with spray dried | | | | |
| strawberry flavor) | | | | |
| Hydrogel Particle | 17.5% | 28.7% | 46.2% | 12.0% |
| (cracker with hydrogel | | | | |
| particle from Example 11) | | | | |

### Example 12—Method of Encapsulating Butter Flavor Using Modified Food Starch as an Emulsifier

TABLE 15
Encapsulated Butter Flavor Composition Prior to Spray Drying

| Materials | Parts |
|---|---|
| Water | 5320 |
| Modified Starch from | 1020 |
| Ingredion | |
| Glucidex 47 | 2160 |
| Faba bean proteins | 1400 |
| Glucono-delta-lactone | 100 |
| IFF Butter Flavor | 1500 |

### Process: Same as in Example 7.

### Experimental Results:

In this formula, the flavor slurry made with modified starch showed flavor droplets with mean size of around 10 μm and viscosity of around 683 Cps at 40 C.

The dry hydrogel particles had a mean particle size of around 89.4 μm and volatile retention of 83.8% with trace amounts of surface oil.

The crackers containing 0.1% neat flavor equivalent made with this sample according to the composition and method of Example 5 also revealed significantly better performance as reported by the trained sensory panel (Table 16). Volatile retention measurement in crackers showed that only gamma decalactone had higher concentration than the benchmark sample (Table 15).

TABLE 16
Time Intensity Sensory Results for Cracker Samples

| | Benchmark sample - | Hydrogel Particle - |
|---|---|---|
| | cracker with spray dried | cracker with hydrogel |
| Time | butter flavor | particle from Example 12 |
| (Seconds) | (LMS) | (LMS) |
| 0 | 0.0 | 0.0 |
| 5 | 0.3 | 0.2 |
| 10 | 0.9 | 1.0 |
| 15 | 2.3 | 2.5 |
| 20 | 4.1 | 4.5 |
| 25 | 5.6 | 6.0 |
| 30 | 6.9 | 7.3 |
| 35 | 7.8 | 8.7 |
| 40 | 8.6 | 9.5 |
| 45 | 9.0 | 10.0 |
| 50 | 9.3 | 10.1 |
| 55 | 9.3 | 10.2 |
| 60 | 9.3 | 10.2 |
| 65 | 9.0 | 9.8 |
| 70 | 8.9 | 9.6 |
| 75 | 8.6 | 9.3 |
| 80 | 8.2 | 9.0 |
| 85 | 7.9 | 8.6 |
| 90 | 7.6 | 8.2 |
| 95 | 7.2 | 7.8 |
| 100 | 6.8 | 7.6 |
| 105 | 6.5 | 7.1 |
| 110 | 6.2 | 6.8 |
| 115 | 5.8 | 6.4 |
| 120 | 5.5 | 5.9 |

TABLE 17
Volatile Retention Results for Cracker Samples (expressed
at % of initial amount of the compound as measured by GC)

| | | decalactone, | decalactone, | |
|---|---|---|---|---|
| | Acetoin | gamma | delta | Total |
| Benchmark sample | 15.1% | 52.5% | 20.3% | 27.7% |
| (cracker with spray | | | | |
| dried butter flavor) | | | | |
| Hydrogel Particle | 11.8% | 55.1% | 17.8% | 26.3% |
| (cracker with | | | | |
| hydrogel particle | | | | |
| from Example 12) | | | | |

### Example 13—Method of Encapsulating Butter Flavor Using Gum Arabic and Tween 60 as Emulsifiers

TABLE 18
Encapsulated Butter Flavor Composition Prior to Spray Drying

| Materials | Parts |
|---|---|
| Water | 5320 |
| Gum Eficacia | 1320 |
| TWEEN 60 from Del Val | 70 |
| Foods | |
| Glucidex 47 | 1790 |
| Faba bean proteins | 1400 |
| Glucono-delta-lactone | 100 |
| IFF Butter Flavor | 1500 |

### Process: Same as in Example 7.

### Experimental Results:

The flavor slurry showed flavor droplets with a mean size of around 5-10 μm and viscosity of around 956 Cps at 40 C.

The dry hydrogel particles had a mean particle size of around 112 μm and volatile retention of 100.4% with trace amounts of surface oil.

The inventive crackers containing 0.1% neat flavor equivalent in this sample made according to the composition and method of Example 5 also revealed significantly higher flavor intensity as reported by the trained sensory panel (Table 19) and higher volatile retention in cracker as measured by GC (Table 20).

TABLE 19
Time Intensity Sensory Results for Cracker Samples

| | Benchmark sample - | Hydrogel Particle - |
|---|---|---|
| | cracker with spray dried | cracker with hydrogel |
| Time | butter flavor | particle from Example 13 |
| (Seconds) | (LMS) | (LMS) |
| 0 | 0.0 | 0.0 |
| 5 | 0.5 | 0.5 |
| 10 | 1.4 | 1.6 |
| 15 | 2.8 | 3.8 |
| 20 | 4.6 | 6.2 |
| 25 | 6.0 | 7.8 |
| 30 | 6.8 | 8.7 |
| 35 | 8.0 | 9.6 |
| 40 | 8.8 | 10.1 |
| 45 | 9.2 | 10.5 |
| 50 | 9.3 | 10.8 |
| 55 | 9.3 | 10.8 |
| 60 | 9.1 | 10.5 |
| 65 | 9.0 | 10.3 |
| 70 | 8.7 | 10.0 |
| 75 | 8.4 | 9.8 |
| 80 | 8.2 | 9.3 |
| 85 | 7.8 | 9.0 |
| 90 | 7.1 | 8.6 |
| 95 | 6.9 | 8.2 |
| 100 | 6.5 | 7.9 |
| 105 | 6.1 | 7.5 |
| 110 | 5.9 | 7.1 |
| 115 | 5.5 | 6.7 |
| 120 | 5.2 | 6.2 |

TABLE 20
Volatile Retention Results for Cracker Samples (expressed
at % of initial amount of the compound as measured by GC)

| | | decalactone, | decalactone, | |
|---|---|---|---|---|
| | Acetoin | gamma | delta | Total |
| Benchmark sample | 14.0% | 50.0% | 18.0% | 26.0% |
| (cracker with spray | | | | |
| dried butter flavor) | | | | |
| Hydrogel Particle | 16.3% | 75.3% | 26.1% | 36.4% |
| (cracker with | | | | |
| hydrogel particle | | | | |
| from Example 13) | | | | |

### Examples 14-16 Comparison Between Hydrogel Encapsulations and Non-Hydrogel Encapsulations

### Example 14—Comparing Prior Art Non-Hydrogel Flavor Encapsulation to Hydrogel Flavor Encapsulation

TABLE 21
Composition of Non-hydrogel butter flavor
encapsulation prior to spray drying

| Material | Parts |
|---|---|
| Faba protein isolate | 8 |
| Maltodextrin maize M150 NGP verified | 71 |
| IFF Butter flavor | 21 |
| Total | 100 |

Process: To approximate a non-hydrogel flavor encapsulation as described in WO14064591 at Table 2, the faba protein isolate was dispersed in water and the pH was adjusted to 3.0 using 0.1M HCl and the solution was stirred overnight at a temperature of 4 C. The maltodextrin was also dispersed in water and stirred overnight at 4 C. To create an emulsion, the protein and maltodextrin solutions were homogenized together with the butter flavor. The emulsion was then spray dried.

TABLE 22
Composition of hydrogel butter flavor
encapsulation prior to spray drying

| Material | Parts |
|---|---|
| GUM EFICACIA | 21.0 |
| GLUCIDEX 47 | 29.8 |
| FABA PROTEIN ISOLATE | 24.0 |
| GLUCONO DELTA LACTONE | 1.20 |
| IFF BUTTER FLAVOR | 24.0 |
| TOTAL | 100 |

### Process: Same as in Example 7.

### Experimental Results:

The non-hydrogel flavor slurry had a viscosity of around 89 Cps at 40 C while the hydrogel flavor slurry viscosity was around 1037 Cps at 40 C.

The dry hydrogel particles had a mean particle size of around 89.1 μm and volatile retention of 100.1% with 0.5% of surface oil. By contrast, the non-hydrogel dry particles had a mean particle size of around 50.9 μm and volatile retention of 89.85% with 3.8% of surface oil.

The inventive, hydrogel crackers containing 0.1% neat flavor equivalent in this example made using the composition and method of Example 5 showed significantly higher flavor intensity as reported by the trained sensory panel as shown in Table 23. Additionally, the hydrogel cracker had higher volatile retention in the cracker as measured by GC (Table 24). This comparison shows the clear distinction between a hydrogel encapsulation and a non-hydrogel encapsulation in a baked good finished product.

TABLE 23
Time Intensity Sensory Results for Cracker Samples

| | Non-hydrogel sample - | |
|---|---|---|
| | cracker with spray | Hydrogel sample - |
| | dried butter flavor | cracker with hydrogel |
| Time | from Table 21 | particle from Table 22 |
| (Seconds) | (LMS) | (LMS) |
| 0 | 0.0 | 0.0 |
| 5 | 1.0 | 1.1 |
| 10 | 1.7 | 2.3 |
| 15 | 2.8 | 3.5 |
| 20 | 4.1 | 5.4 |
| 25 | 5.0 | 6.6 |
| 30 | 5.8 | 7.4 |
| 35 | 6.4 | 8.0 |
| 40 | 6.9 | 8.3 |
| 45 | 7.2 | 8.7 |
| 50 | 7.2 | 8.8 |
| 55 | 7.3 | 8.7 |
| 60 | 7.2 | 8.7 |
| 65 | 6.9 | 8.4 |
| 70 | 6.6 | 8.2 |
| 75 | 6.3 | 7.8 |
| 80 | 6.1 | 7.6 |
| 85 | 5.9 | 7.3 |
| 90 | 5.7 | 7.1 |
| 95 | 5.5 | 6.7 |
| 100 | 5.1 | 6.5 |
| 105 | 4.9 | 6.1 |
| 110 | 4.6 | 5.8 |
| 115 | 4.3 | 5.5 |
| 120 | 4.0 | 5.4 |

TABLE 24
Volatile Retention Results for Cracker Samples
(expressed at % of initial amount of the compound as measured by GC)

| | | decalactone, | decalactone, | |
|---|---|---|---|---|
| | Acetoin | gamma | delta | Total |
| Hydrogel sample - | 15.2% | 95.5% | 40.7% | 43.2% |
| cracker with | | | | |
| hydrogel particle | | | | |
| from Table 22 | | | | |
| Non-hydrogel sample - | 14.5% | 56.5% | 26.8% | 29.0% |
| cracker with spray | | | | |
| dried butter flavor | | | | |
| from Table 21 | | | | |

### Example 15—Comparing Non-Hydrogel Flavor Encapsulation to Hydrogel Flavor Encapsulation at a 24% Payload Level

TABLE 25
Composition of hydrogel butter flavor
encapsulation prior to spray drying

| Material | Parts |
|---|---|
| GUM EFICACIA | 21.0 |
| GLUCIDEX 47 | 29.8 |
| FABA PROTEIN ISOLATE | 24.0 |
| GLUCONO DELTA LACTONE | 1.20 |
| IFF BUTTER FLAVOR | 24.0 |
| TOTAL | 100 |

Process: For the hydrogel encapsulation sample, the process was the same as in Example 7. For the non-hydrogel encapsulation sample, steps 4 and 5 from the process in Example 7 were eliminated and the slurry was spray dried as in step 6.

### Experimental Results:

The non-hydrogel flavor slurry had a viscosity of around 450 Cps at 40 C while the hydrogel flavor slurry viscosity was around 1037 Cps at 40 C.

The dry hydrogel particles had a mean particle size of around 89.1 μm and volatile retention of 100.1% with 0.5% of surface oil. By contrast, the non-hydrogel dry particles had a mean particle size of around 204.0 μm and volatile retention of 94.75% with 0.7% of surface oil.

The inventive, hydrogel crackers containing 0.1% neat flavor equivalent in this example showed significantly higher flavor intensity as reported by the trained sensory panel as shown in Table 26. Additionally, the hydrogel cracker had higher volatile retention in the cracker as measured by GC (Table 27). This comparison shows another clear distinction between a hydrogel encapsulation and a non-hydrogel encapsulation in a baked good finished product.

TABLE 26
Time Intensity Sensory Results for Cracker Samples

| | 24% Butter | 24% Butter |
|---|---|---|
| Time | Hydrogel | Non-Hydrogel |
| (Seconds) | Particle | Particle |
| 0 | 0.0 | 0.0 |
| 5 | 1.0 | 0.8 |
| 10 | 2.7 | 1.7 |
| 15 | 4.6 | 3.3 |
| 20 | 6.1 | 4.8 |
| 25 | 7.2 | 6.1 |
| 30 | 7.7 | 6.7 |
| 35 | 8.4 | 7.3 |
| 40 | 8.9 | 7.7 |
| 45 | 9.2 | 8.0 |
| 50 | 9.4 | 8.0 |
| 55 | 9.3 | 8.0 |
| 60 | 9.2 | 7.9 |
| 65 | 8.8 | 7.5 |
| 70 | 8.3 | 7.2 |
| 75 | 8.1 | 6.9 |
| 80 | 7.8 | 6.5 |
| 85 | 7.6 | 6.2 |
| 90 | 7.1 | 5.9 |
| 95 | 6.8 | 5.6 |
| 100 | 6.4 | 5.3 |
| 105 | 6.2 | 4.9 |
| 110 | 5.9 | 4.7 |
| 115 | 5.6 | 4.5 |
| 120 | 5.4 | 4.2 |

TABLE 27
Volatile Retention Results for Cracker Samples
(expressed at % of initial amount of the compound as measured by GC)

| | | decalactone, | decalactone, | |
|---|---|---|---|---|
| | Acetoin | gamma | delta | Total |
| 24% Butter Hydrogel | 15.1% | 80.1% | 43.0% | 38.5% |
| Particle | | | | |
| 24% Butter Non- | 12.0% | 50.7% | 20.9% | 25.1% |
| Hydrogel Particle | | | | |
| W/O heating | | | | |

### Example 16—Comparing Non-Hydrogel Flavor Encapsulation to Hydrogel Flavor Encapsulation at a 40% Payload Level

TABLE 28
Composition of hydrogel butter flavor
encapsulation prior to spray drying

| MATERIAL | PARTS |
|---|---|
| GUM EFICACIA | 21.0 |
| GLUCIDEX 47 | 13.8 |
| FABA PROTEIN ISOLATE | 24.0 |
| GLUCONO DELTA LACTONE | 1.20 |
| IFF BUTTER FLAVOR | 40.0 |
| TOTAL | 100 |

Process: For the hydrogel encapsulation sample, the process was the same as in Example 7. For the non-hydrogel encapsulation sample, steps 4 and 5 from the process in Example 7 were eliminated and the slurry was spray dried as in step 6.

### Experimental Results:

The non-hydrogel flavor slurry had a viscosity of around 406 Cps at 40 C while the hydrogel flavor slurry viscosity was around 880 Cps at 40 C.

The dry hydrogel particles had a mean particle size of around 95.0 μm and volatile retention of 101.1% with 2.9% of surface oil. By contrast, the non-hydrogel dry particles had a mean particle size of around 129.0 μm and volatile retention of 99.5% with 2.9% of surface oil.

The inventive, hydrogel crackers containing 0.1% neat flavor equivalent in this example made according to the composition and method of Example 5 again showed significantly higher flavor intensity as reported by the trained sensory panel as shown in Table 29. Additionally, the hydrogel cracker had higher volatile retention in the cracker as measured by GC (Table 30). This comparison shows another clear distinction between a hydrogel encapsulation and a non-hydrogel encapsulation in a baked good finished product.

TABLE 29
Time Intensity Sensory Results for Cracker Samples

| | 40% Butter | 40% Butter |
|---|---|---|
| Time | Hydrogel | Non-Hydrogel |
| (Seconds) | Particle | Particle |
| 0 | 0.0 | 0.0 |
| 5 | 0.9 | 0.9 |
| 10 | 2.1 | 2.1 |
| 15 | 3.4 | 3.4 |
| 20 | 4.7 | 4.9 |
| 25 | 6.0 | 5.8 |
| 30 | 7.0 | 6.5 |
| 35 | 7.6 | 6.9 |
| 40 | 8.0 | 7.4 |
| 45 | 8.1 | 7.4 |
| 50 | 8.3 | 7.3 |
| 55 | 8.3 | 7.4 |
| 60 | 8.0 | 7.3 |
| 65 | 7.7 | 7.0 |
| 70 | 7.6 | 6.8 |
| 75 | 7.3 | 6.4 |
| 80 | 6.9 | 6.1 |
| 85 | 6.7 | 5.8 |
| 90 | 6.5 | 5.6 |
| 95 | 6.0 | 5.3 |
| 100 | 5.7 | 5.2 |
| 105 | 5.3 | 4.8 |
| 110 | 5.1 | 4.5 |
| 115 | 4.8 | 4.4 |
| 120 | 4.5 | 4.1 |

TABLE 30
Volatile Retention Results for Cracker Samples
(expressed at % of initial amount of the compound as measured by GC)

| | | decalactone, | decalactone, | |
|---|---|---|---|---|
| | Acetoin | gamma | delta | Total |
| 40% Butter Hydrogel | 15.5% | 107.1% | 62.5% | 49.3% |
| Particle | | | | |
| 40% Butter Non- | 10.9% | 58.2% | 22.0% | 26.9% |
| Hydrogel Particle | | | | |
| W/O Heating | | | | |

### Example 17. Method of Encapsulating Butter Flavor with Cross-Linking Agent

TABLE 31
Encapsulated Butter Flavor Composition Prior to vacuum drying

| Materials | Parts | % |
|---|---|---|
| Water | 5320 | 45.2 |
| Emulsifier - gum arabic with trade name Gum | 1320 | 11.2 |
| Eficacia ™ from Nexira | | |
| Carrier - spray dried glucose syrup with trade | 1860 | 15.8 |
| name Glucidex 47 ™ from Roquette | | |
| Protein - Faba bean protein with >85% protein | 1500 | 12.7 |
| content from AGT foods | | |
| Cross-linker- Calcium Chloride | 270 | 2.3 |
| Flavor - Butter Flavor from IFF | 1500 | 12.7 |
| Total | 11770 | 100 |

### Process:

1. The emulsifier and carrier were dissolved in ambient temperature water using an overhead mixer by stirring at 500 rpm for at least one hour until the solids were fully dissolved.
2. The flavor oil was mixed with the emulsifier/carrier solution and homogenized using a Silverson Verso in-line rotor-stator mixer operating at 2500 or 3450 rpm and a circulation rate of 1 kilogram per minute.
3. The homogenized slurry was then heated to about 60 C followed by addition of Calcium Chloride and mixed for 15 min. Then faba proteins were added into the slurry and mixed until fully dissolved.
4. The slurry was then heated to 75-80 C and held for at least 30 minutes with continuous mixing.
5. The slurry was then cooled to ambient temperature prior to drying using a pilot-scale vacuum dryer at 70 C for around 7 hours or until fully dried.
6. The vacuum dried encapsulated flavor particles then milled using COMIL unit to achieve desired particle size range and then stored in sealed aluminum bag at −5° C. until analysis.

### Experimental Results:

The paste hydrogel flavor slurry prior to drying step showed flavor droplets with mean size of 1.90 μm and viscosity of around 3725 cps at 40° C.

The vacuum dried butter-flavored hydrogel particles sieved to achieve particle size ranging from 250-1200 μm and volatile retention of 97.1% and free oil of 1.96% indicating strong encapsulation efficiency.

The performance of butter hydrogel particles was evaluated in cracker as a model of baked products. The cracker was made as shown in Example 5. The sensory performance of hydrogel particles was assessed against benchmark (i.e. not hydrogel) spray dried flavor at the same active level of flavor oil (i.e. iso-active flavor level) in the cracker. Table 32 shows the trained panel sensory results and Table 33 shows measured volatile retention in crackers. Both cracker samples contained as iso-active butter flavor level of 0.1% neat oil equivalent. Both data sets show that the inventive butter hydrogel particle outperformed the spray dried comparative benchmark sample by having significantly higher flavor intensity in sensory testing and higher retained actives in cracker as measured by chromatography technique. The results illustrated that encapsulating flavoring compounds in hydrogel structure enhanced the active protection during dough preparation and baking conditions resulting in a finished product with enhanced sensory performance.

TABLE 32
Time Intensity Sensory Results for Cracker Samples

| | | Hydrogel |
|---|---|---|
| | Benchmark | Particle - |
| | sample - cracker | cracker with |
| | with spray dried | hydrogel particle |
| Time | butter flavor | from Example 17 |
| (Seconds) | (LMS) | (LMS) |
| 0 | 0.0 | 0.0 |
| 5 | 1.5 | 1.9 |
| 10 | 3.2 | 3.6 |
| 15 | 4.4 | 5.2 |
| 20 | 5.3 | 6.2 |
| 25 | 5.9 | 7.0 |
| 30 | 6.4 | 7.5 |
| 35 | 6.8 | 8.1 |
| 40 | 7.3 | 8.6 |
| 45 | 7.5 | 9.0 |
| 50 | 7.6 | 9.1 |
| 55 | 7.7 | 9.0 |
| 60 | 7.5 | 8.9 |
| 65 | 7.0 | 8.7 |
| 70 | 6.5 | 8.1 |
| 75 | 6.2 | 7.5 |
| 80 | 6.1 | 7.1 |
| 85 | 5.9 | 6.8 |
| 90 | 5.6 | 6.5 |
| 95 | 5.3 | 6.3 |
| 100 | 4.9 | 5.9 |
| 105 | 4.7 | 5.3 |
| 110 | 4.3 | 5.0 |
| 115 | 4.0 | 4.6 |
| 120 | 3.7 | 4.3 |

TABLE 33
Volatile Retention Results for Cracker Samples
(expressed at % of initial amount of the compound as measured by GC)

| | | decalactone, | decalactone, | |
|---|---|---|---|---|
| | Acetoin | gamma | delta | Total |
| Benchmark sample | 11.6% | 43.4% | 19.1% | 24.6% |
| (cracker with spray dried | | | | |
| butter flavor) | | | | |
| Vacuum Dried | 14.8% | 71.3% | 34.3% | 33.7% |
| Hydrogel Particle | | | | |
| (cracker with hydrogel | | | | |
| particle from Example | | | | |
| 17) | | | | |

### Example 18. Method of Encapsulating Butter Flavor with Gelling Agent

TABLE 34
Encapsulated Butter Flavor Composition Prior to vacuum drying

| Materials | Parts | % |
|---|---|---|
| Water | 5320 | 45.5 |
| Emulsifier - gum arabic with trade name Gum | 1320 | 11.3 |
| Eficacia ™ from Nexira | | |
| Carrier - spray dried glucose syrup with trade | 1860 | 15.9 |
| name Glucidex 47 ™ from Roquette | | |
| Protein - Faba bean protein with >85% protein | 1500 | 12.8 |
| content from AGT foods | | |
| Gelling agent- Advanta Gel Starch from | 200 | 1.7 |
| Ingredion | | |
| Flavor - Butter Flavor from IFF | 1500 | 12.8 |
| Total | 11,700 | 100 |

### Process:

1. The emulsifier and carrier were dissolved in ambient temperature water using an overhead mixer by stirring at 500 rpm for at least one hour until the solids were fully dissolved.
2. The flavor oil was mixed with the emulsifier/carrier solution and homogenized using a Silverson Verso in-line rotor-stator mixer operating at 2500 or 3450 rpm and a circulation rate of 1 kilogram per minute.
3. The homogenized slurry was then heated to about 60 C followed by addition of Advanta Gel Starch and mixed until fully dissolved. Then faba proteins were added into the slurry and mixed until fully dissolved.
4. The slurry was then heated to 75-80 C and held for at least 30 minutes with continuous mixing.
5. The slurry was then cooled to ambient temperature prior to drying using a pilot-scale vacuum dryer at 70 C for around 7 hours or until fully dried.
6. The vacuum dried encapsulated flavor particles then milled using COMIL unit to achieve desired particle size range and then stored in sealed aluminum bag at −5° C. until analysis.

### Experimental Results:

The paste hydrogel flavor slurry prior to drying step showed flavor droplets with mean size of 2.4 μm and viscosity of around 4350 cps at 40° C.

The vacuum dried butter-flavored hydrogel particles sieved to achieve particle size ranging from 250-1200 μm and volatile retention of 87.6% and free oil of 0.98% indicating strong encapsulation efficiency.

The performance of butter hydrogel particles was evaluated in cracker as a model of baked products. The cracker was made as shown in Example 5. The sensory performance of hydrogel particles was assessed against benchmark (i.e. not hydrogel) spray dried flavor at the same active level of flavor oil (i.e. iso-active flavor level) in the cracker. Table 35 shows the trained panel sensory results and Table 36 shows measured volatile retention in crackers. Both cracker samples contained as iso-active butter flavor level of 0.1% neat oil equivalent. Both data sets show that the inventive butter hydrogel particle outperformed the spray dried comparative benchmark sample by having significantly higher flavor intensity in sensory testing and higher retained actives in cracker as measured by chromatography technique. The results illustrated that encapsulating flavoring compounds in hydrogel structure enhanced the active protection during dough preparation and baking conditions resulting in a finished product with enhanced sensory performance.

TABLE 35
Time Intensity Sensory Results for Cracker Samples

| | | Hydrogel |
|---|---|---|
| | Benchmark | Particle - |
| | sample - cracker | cracker with |
| | with spray dried | hydrogel particle |
| Time | butter flavor | from Example 18 |
| (Seconds) | (LMS) | (LMS) |
| 0 | 0.0 | 0.0 |
| 5 | 0.7 | 0.6 |
| 10 | 1.7 | 1.7 |
| 15 | 2.8 | 3.1 |
| 20 | 4.0 | 4.2 |
| 25 | 5.2 | 5.8 |
| 30 | 6.2 | 6.8 |
| 35 | 6.9 | 7.8 |
| 40 | 7.2 | 8.4 |
| 45 | 7.5 | 8.9 |
| 50 | 7.6 | 8.9 |
| 55 | 7.4 | 8.8 |
| 60 | 7.3 | 8.6 |
| 65 | 7.1 | 8.5 |
| 70 | 7.0 | 8.3 |
| 75 | 6.8 | 7.9 |
| 80 | 6.6 | 7.7 |
| 85 | 6.2 | 7.6 |
| 90 | 6.1 | 7.3 |
| 95 | 5.9 | 6.9 |
| 100 | 5.8 | 6.7 |
| 105 | 5.4 | 6.5 |
| 110 | 5.2 | 6.3 |
| 115 | 5.1 | 5.9 |
| 120 | 4.9 | 5.8 |

TABLE 36
Volatile Retention Results for Cracker Samples
(expressed at % of initial amount of the compound as measured by GC)

| | | decalactone, | decalactone, | |
|---|---|---|---|---|
| | Acetoin | gamma | delta | Total |
| Benchmark sample | 11.6% | 43.4% | 13.7% | 24.6% |
| (cracker with spray dried | | | | |
| butter flavor) | | | | |
| Vacuum Dried Hydrogel | 11.6% | 57.1% | 24.4% | 27.9% |
| Particle | | | | |
| (cracker with hydrogel | | | | |
| particle from Example | | | | |
| 18) | | | | |

What is claimed is:

1. A method of encapsulation comprising the steps of homogenizing an emulsifier with an active and a carrier to create an emulsified active, heating the emulsified active to a first temperature of at least 35 C, mixing the heated emulsified active with a protein, and further heating the mixture to a second temperature of at least 50 C to create a hydrogel.

2. The method as in claim 1, further comprising a step of holding the hydrogel at the second temperature for a gelation time of at least 30 minutes.

3. The method as in claim 1, further comprising a step of cooling the hydrogel to a cooling temperature of from about 50 C to about 70 C.

4. The method as in claim 1, further comprising a step of drying the hydrogel to create hydrogel particles.

5. The method as in claim 4, wherein the hydrogel particles have a particle size of from about 2 microns to about 500 microns.

6. The method as in claim 1, wherein the protein comprises at least one of a chickpea protein, a lentil protein, a faba bean protein, a soybean protein, or combinations thereof.

7. The method as in claim 1, wherein the protein is an unhydrolyzed faba bean protein.

8. The method as in claim 1, wherein the protein has a protein content of at least 85% w/w by weight of the protein.

9. The method as in claim 1, wherein the method further comprises mixing the active with a weighting agent prior to the homogenization step.

10. The method as in claim 1, wherein the active is present in an amount of from about 0.1% to about 60% w/v by weight of the hydrogel.

11. The method as in claim 4, wherein the active is present in an amount of from about 0.1% to about 60% w/w by weight of the hydrogel particles.

12. The method as in claim 1, wherein the protein is present in an amount of from about 7% to about 20% w/v by weight of the hydrogel.

13. The method as in claim 4, wherein the protein is present in an amount of from about 10% to about 50% w/w by weight of the hydrogel particles.

14. The method as in claim 1, wherein the method further comprises the step of mixing the emulsified active with a gelling agent, a cross-linking agent or a combination thereof prior to mixing the emulsified active with the protein.

15. A microcapsule comprising an active and a shell, wherein the active comprises a flavor, the shell comprises a faba bean protein, and the microcapsule has a volatile retention of at least 80%.

16. The microcapsule as in claim 15, wherein the flavor is present in an amount of from about 0.1% to about 60% w/w by weight of the microcapsule.

17. A product comprising a product base and a microcapsule, wherein the microcapsule comprises an active and a shell, wherein the shell comprises a faba bean protein, and wherein the microcapsule has a volatile retention of at least 80%.

18. The product as in claim 17, wherein the product has a higher flavor intensity score as measured by sensory testing than an iso-active flavored product without the microcapsule.

19. The product as in claim 17, wherein the active is present in the microcapsule in an amount of from about 0.1% to about 60% w/w by weight of the microcapsule.

20. The product as in claim 17, wherein the microcapsule is present in an amount of from about 0.1% to about 10% w/w by weight of the product.
